(19) United States
(12) Patent Application Publication
(10) Pub. No.: US 2024/0382872 A1
(43) Pub. Date: Nov. 21, 2024

(54) GAS REMOVAL SYSTEM AND METHODS

(71) Applicant: AILNH, LLC, Cedar Knolls, NJ (US)

(72) Inventors: Norton Herrick, Boca Raton, FL (US); David Saloff, Pacific Palisades, CA (US); Miles Herrick, New York, NY (US); William F. Ryczek, Pomona, CA (US); Thomas Wang, Alhambra, CA (US)

(21) Appl. No.: 18/637,418

(22) Filed: Apr. 16, 2024

Related U.S. Application Data
(60) Provisional application No. 63/466,407, filed on May 15, 2023.

(51) Int. Cl. B01D 19/00; A61M 5/38; B01D 39/16

(52) U.S. Cl. CPC B01D 19/0031; A61M 5/38; B01D 39/16

(57) ABSTRACT

This disclosure is directed to devices and fluid delivery systems for separating a gas from a fluid. The device includes a filter and a body. The body defines an interior and the filter is within the interior and segments the interior into a first chamber and a second chamber. The device includes an inlet for communicating the fluid into the first chamber; a first outlet for communicating the fluid out of the first chamber; and a second outlet for communicating the gas out of the second chamber. The filter is impermeable to liquid and isolates the second chamber from liquid. The filter is permeable to gas and permits the gas to flow from the first chamber to the second chamber to separate the gas from the liquid of the fluid.

### CROSS REFERENCE TO RELATED APPLICATIONS

This application claims the benefit of U.S. Provisional Patent App. No. 63/466,407, filed May 15, 2023, the disclosure of which is hereby incorporated by reference herein.

### TECHNICAL FIELD

This disclosure is generally directed to the field of fluid delivery, and more particularly to systems and methods for removing air bubbles or other gases from fluids flowing through a fluid delivery system.

### BACKGROUND

Gas bubbles traveling through fluid delivery systems can cause significant problems. Fluid delivery systems for delivering fluid to patients can be vulnerable to gas bubbles in the fluids, and other fluid delivery systems in the automotive, aerospace, or other industries can also be vulnerable to problems caused by gas bubbles in fluids. For example, gases can be introduced into blood vessels during surgery or other medical procedures. Gas can be introduced in the form of bubbles trapped in a fluid introduced into the blood vessel (e.g., a blood transfusion, an intravenous (IV) fluid line supplying a fluid such as a saline solution or medicine). Small air bubbles may be present in the fluid as supplied. Additional air bubbles may be formed, for example, when priming the IV line if a roller clamp is released too quickly when priming the line.

An air embolism can occur when an air bubble or embolus delivered with an intravenous fluid becomes trapped in a blood vessel or in the heart and obstructs the normal flow of blood through the blood vessel (e.g., a vascular air embolus (VAE)) or the heart. Air in a patient's veins can travel to the right side of the heart and from the heart to the lungs. Air trapped in vessels providing blood to the lungs can inhibit pulmonary circulation and cause chest pain and rapid breathing. In some patients, the air may pass to the left side of the heart and on to the brain or the coronary arteries, which can lead to more serious complications. The effect of an air embolism is directly related to the size of the embolus and the rate of entry of the air into the blood vessel. 50 ml of air cause hypotension and dysrhythmias, while 300 ml can cause death if introduced rapidly, generally due to circulatory obstruction and cardiovascular collapse.

A pump can be used to control the rate at which the fluids are introduced. Such pumps may include a system to detect when air is in the IV line. If an air bubble reaches the pump, an alarm can sound to alert the nursing staff or other caregiver and the pump turns off. The caregiver then must go to the patient, attempt to remove the air bubbles from the IV line. Every health care facility may have a specific protocol for this procedure, but it can involve low-tech and/or time-consuming solutions such as “flicking” the IV bag and/or IV line to try and release the bubbles and get them to collect at the top of the bag, away from outlet.

Significant financial and human resources are expended to prime IV lines, reset the pump alarm when the alarm goes off, and purge air from the IV line. Additionally, the alarm built into the pump is a disturbance to the patient, as it is likely to wake the patient every time it goes off.

Accordingly, there exists an unmet need to remove gas from lines in fluid delivery systems.

### SUMMARY

There exists a need to remove gas from lines in fluid delivery systems without substantially impacting the flow rate of the fluid flowing through the fluid delivery systems. These needs are met, to a great extent, by a device for separating a gas from a fluid in accordance with aspects of this invention. The device includes a filter and a body defining an interior. The filter is within the interior and segments the interior into a first chamber and a second chamber. The device also includes an inlet that is configured to communicate the fluid into the first chamber and a first outlet that is configured to communicate the fluid out of the first chamber. The device also includes a second outlet that is configured to communicate the gas out of the second chamber. The filter is impermeable to a liquid of the fluid and is configured to isolate the second chamber from the liquid of the fluid when the fluid is in the first chamber. The filter is permeable to the gas of the fluid and is configured to permit communication of the gas from the first chamber to the second chamber to separate the gas from the liquid of the fluid.

Implementations may include one or more of the following features. The device where the first outlet and the second outlet extend from a top of the body. The second outlet is larger than the first outlet. A center of each the first outlet and the second outlet are each laterally offset from a center of the top of the body. The inlet has an inner diameter and the first outlet has an inner diameter. The inner diameter of the first outlet is less than the inner diameter of the inlet. An inner surface of a top of the body tapers towards the second outlet. An inner surface of a bottom of the body tapers towards the first outlet. The second outlet may include a bottom surface that slopes downwardly away from the second chamber. The body may include projections that extend into the second chamber to resist a pressure exerted by the fluid on the filter. The projections extend radially inwardly into the second chamber and intersect at a central portion of the second chamber such that the projections define a star-shaped pattern. The projections extend perpendicular to the filter and segment the second chamber into a plurality of sub-chambers. The second outlet may include a plurality of vents and each vent of the plurality of vents fluidly communicates with at least one respective sub-chamber of the plurality of sub-chambers. The first outlet of the device is configured to connect the device to an inlet of a pump. The first outlet of the device is configured to connect the device to the inlet of the pump via a supply line. The inlet of the device is configured to connect the device to an outlet of a pump. The inlet of the device is configured to connect the device to the outlet of the pump via a supply line. The filter is disk shaped. The filter is a membrane. The membrane may include at least one of PTFE or acrylic copolymer matrix. The membrane has a water entry pressure greater than 750 mbar. The body may include a first portion and a second portion, and the filter is sandwiched between the first portion and the second portion. The filter is a first filter, and the device may include a second filter held within the second chamber. The second filter is impermeable to the liquid of the fluid, and the filter is permeable to the gas of the fluid. The pad is configured to absorb the liquid and the pad is configured to be permeable to the gas. The device may include a valve that is configured to selectively isolate the filter from the fluid in the first chamber. The valve is configured to automatically isolate the filter from the fluid in the first chamber when at least one of the fluid fills a volume of the first chamber or the fluid exerts a predetermined pressure on the valve.

Another general aspect of the invention is directed to a fluid delivery system that includes a fluid source that is configured to contain a fluid having a liquid and a gas. The fluid delivery system also includes a fluid destination that is configured to receive fluid from the fluid source. The fluid delivery system also includes supply lines that fluidly connect the fluid source to the fluid destination and that are configured to convey the fluid from the fluid source to the fluid destination at a flow rate. The fluid delivery system also includes a gas removal device that is fluidly connected to the supply lines between the fluid source and the fluid destination. The device is configured to separate the gas from the fluid without restricting the flow rate of the fluid through the supply lines.

The fluid delivery system where the gas removal device may include an inlet fluidly connected to an inlet supply line of the supply lines. The inlet supply line has an inner diameter, the inlet has an inner diameter, and the inner diameter of the inlet is greater than or equal to the inner diameter of the inlet supply line. The inner diameter of the inlet is a first inner diameter of the inlet, and the inlet has a second inner diameter. The inlet supply line has an outer diameter, and the second inner diameter of the inlet is greater than or equal to the outer diameter of the inlet supply line. The second inner diameter of the inlet is greater than the first inner diameter of the inlet. The gas removal device may include a body that defines an interior, and the first inner diameter of the inlet is between the interior and the second inner diameter of the inlet. The gas removal device may include an outlet fluidly connected to an outlet supply line of the supply lines. The outlet supply line has an inner diameter, the outlet has an inner diameter, and the inner diameter of the outlet is less than or equal to the inner diameter of the outlet supply line. The inner diameter of the outlet is a first inner diameter of the outlet, and the outlet has a second inner diameter. The outlet supply line has an outer diameter, and the second inner diameter of the outlet is greater than or equal to the outer diameter of the outlet supply line. The second inner diameter of the outlet is greater than the first inner diameter of the outlet. The gas removal device may include a body that defines an interior and the first inner diameter of the outlet is between the interior and the second inner diameter of the outlet. The fluid destination has a first cross-sectional flow area through which the fluid is configured to flow, the outlet supply line defines a second cross-sectional flow area through which the fluid is configured to flow, and the first inner diameter of the outlet defines a third cross-sectional flow area through which the fluid is configured to flow. The third cross-sectional flow area is greater than the first cross-sectional flow area and is less than the second cross-sectional flow area. The fluid delivery system may include a pump that is fluidly connected to the supply lines and that is configured to pump the fluid from the fluid source to the fluid destination through the supply lines and through the gas removal device. The device is upstream from the pump. The device is downstream from the pump. When the fluid delivery system is not primed, the device is configured to separate the gas from the fluid without restricting the flow rate of the fluid through the supply lines. When the fluid delivery system is primed, the gas removal device is configured to separate the gas from the fluid without restricting the flow rate of the fluid through the supply lines. The gas removal device may include a filter and a body defining an interior. The filter is within the interior and segments the interior into a first chamber and a second chamber. The gas removal device may include an inlet that is configured to communicate the fluid into the first chamber and a first outlet that is configured to communicate the fluid out of the first chamber. The gas removal device may include a second outlet that is configured to communicate the gas out of the second chamber. The filter is impermeable to a liquid of the fluid and is configured to isolate the second chamber from the liquid of the fluid when the fluid is in the first chamber. The filter is permeable to the gas of the fluid and is configured to permit communication of the gas from the first chamber to the second chamber to separate the gas from the liquid of the fluid.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1A shows a schematic view of a first embodiment of a fluid delivery system;

FIG. 1B shows a schematic view of a second embodiment of a fluid delivery system;

FIG. 1C shows a schematic view of a third embodiment of the fluid delivery system;

FIG. 2 shows a schematic view of a fourth embodiment of a fluid delivery system;

FIG. 3 shows a perspective view of a first embodiment of a device for removing gas from a fluid;

FIG. 4 shows an exploded view of the first embodiment of the device;

FIG. 5 shows a top view of the first embodiment of the device;

FIG. 6 shows a cross-section view along section line 6-6 of the device shown in FIG. 5;

FIG. 7 shows a cross-section view along section line 7-7 of the device shown in FIG. 6;

FIG. 8 shows a perspective view of a second embodiment of a device for removing gas from a fluid;

FIG. 9 shows an exploded view of the second embodiment of the device;

FIG. 10 shows a top view of the second embodiment of the device;

FIG. 11 shows a cross-section view along section line 11-11 of the device shown in FIG. 10;

FIG. 12 shows a cross-section view along section line 12-12 of the device shown in FIG. 11;

FIG. 13 shows a perspective view of a third embodiment of a device for removing gas from a fluid;

FIG. 14 shows an exploded view of the third embodiment of the device;

FIG. 15 shows a side view of the third embodiment of the device;

FIG. 16 shows a cross-section view along section line 16-16 of the device shown in FIG. 15;

FIG. 17 shows a cross-section view along section line 17-17 of the device shown in FIG. 16;

FIG. 18 shows a perspective view of a fourth embodiment of a device for removing gas from a fluid;

FIG. 19 shows an exploded view of the fourth embodiment of the device;

FIG. 20 shows a side view of the fourth embodiment of the device;

FIG. 21 shows a cross-section view along section line 21-21 of the device shown in FIG. 20;

FIG. 22 shows a cross-section view along section line 22-22 of the device shown in FIG. 21;

FIG. 23 shows a perspective view from an inlet-side of a fifth embodiment of the device for removing gas from a fluid;

FIG. 24 shows a perspective view from an outlet-side of the fifth h embodiment of the device;

FIG. 25 shows a cross-section view along section line 25-25 of the device shown in FIG. 24;

FIG. 26 shows a perspective view from an inlet-side of a sixth embodiment of the device for removing gas from a fluid;

FIG. 27 shows a perspective view from an outlet-side of the sixth embodiment of the device;

FIG. 28 shows a cross-section view along section line 28-28 of the device shown in FIG. 27;

FIG. 29 shows a perspective view from an inlet-side of a seventh embodiment of the device for removing gas from a fluid;

FIG. 30 shows a perspective view from an outlet-side of the seventh embodiment of the device;

FIG. 31 shows a cross-section view along section line 31-31 of the device shown in FIG. 30;

FIG. 32 shows a perspective view of an eighth embodiment of the device for removing gas from a fluid;

FIG. 33 shows a perspective view from an outlet-side of the eighth embodiment of the device;

FIG. 34 shows a cross-section view along section line 34-34 of the device shown in FIG. 33;

FIG. 35 is a top, front and right side perspective view of the first embodiment of the device for removing gas from a fluid;

FIG. 36 is a front elevational view of the first embodiment;

FIG. 37 is a rear elevational view of the first embodiment;

FIG. 38 is a right side elevational view of the first embodiment;

FIG. 39 is a left side elevational view of the first embodiment;

FIG. 40 is a top plan view of the first embodiment;

FIG. 41 is a bottom plan view of the first embodiment;

FIG. 42 is a top, front and right side perspective view of the second embodiment of the device for removing gas from a fluid;

FIG. 43 is a front elevational view of the second embodiment;

FIG. 44 is a rear elevational view of the second embodiment;

FIG. 45 is a right side elevational view of the second embodiment;

FIG. 46 is a left side elevational view of the second embodiment;

FIG. 47 is a top plan view of the second embodiment;

FIG. 48 is a bottom plan view of the second embodiment;

FIG. 49 is a top, front and right side perspective view of the third embodiment of the device for removing gas from a fluid;

FIG. 50 is a front elevational view of the third embodiment;

FIG. 51 is a rear elevational view of the third embodiment;

FIG. 52 is a right side elevational view of the third embodiment;

FIG. 53 is a left side elevational view of the third embodiment;

FIG. 54 is a top plan view of the third embodiment;

FIG. 55 is a bottom plan view of the third embodiment;

FIG. 56 is a top, front and right side perspective view of the fourth embodiment of the device for removing gas from a fluid;

FIG. 57 is a front elevational view of the fourth embodiment;

FIG. 58 is a rear elevational view of the fourth embodiment;

FIG. 59 is a right side elevational view of the fourth embodiment;

FIG. 60 is a left side elevational view of the fourth embodiment;

FIG. 61 is a top plan view of the fourth embodiment; and

FIG. 62 is a bottom plan view of the fourth embodiment.

### DETAILED DESCRIPTION

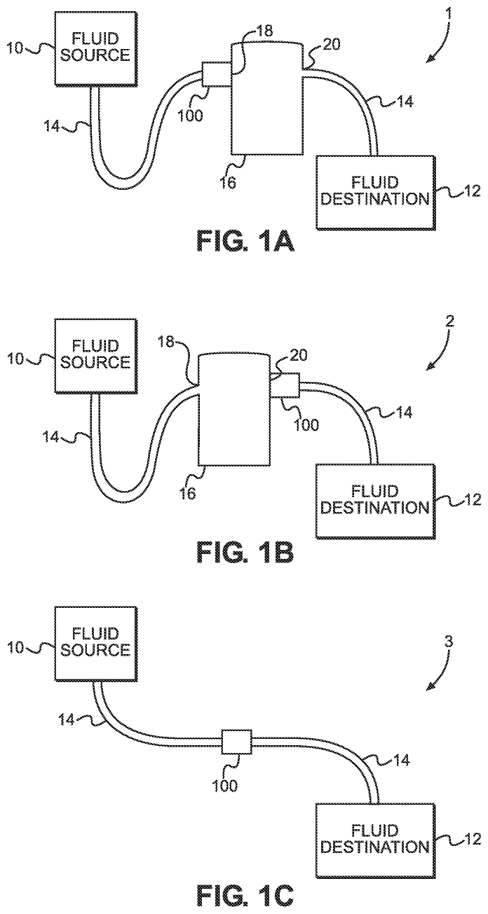

FIG. 1A shows a schematic view of a fluid delivery system 1 according to aspects of the invention. The fluid delivery system 1 can include a fluid source 10 and a fluid destination 12 and the fluid delivery system 1 can transport a fluid from the fluid source 10 to the fluid destination 12. The fluid can be any flowable substance or combination of substances and includes some substances in a liquid state. Example fluids can include water with or without dissolved substances therein, gasoline or other fuel source, among other possibilities. The fluid source 10 can be a source of fluid including any a bag, tank, pipe, container or other structure that hold and/or convey the fluid. The fluid destination can be any structure or entity that can receive the fluid including for example a container, an engine, a patient, among other possibilities. The fluid delivery system 1 can include supply lines 14 such as for example tubes, pipes, hoses, conduits, functional equivalents, and combinations thereof. The supply lines 14 can fluidly connect the fluid source 10 to the fluid destination 12 to transport the fluid from the fluid source 10 to the fluid destination 12.

In embodiments, the fluid delivery system 1 can include a device that can agitate the fluid between the fluid source 10 and the fluid destination 12 to generate gas bubble within the fluid. The device that can agitate the fluid can be, for example, a pump 16, though other devices are possible such as a valve, port, or any number of other devices. Accordingly, the term pump 16 is not limited to strictly to pumps and can include any number of other device that can agitate the fluid to generate air bubbles.

The pump 16 can be connected to the supply lines 14 between the fluid source 10 and the fluid destination 12 and can pump fluid from the fluid source 10 to the fluid destination 12 through the supply lines 14. In embodiments, the pump 16 can monitor and/or control the flow rate of the fluid through the fluid delivery system 1. In embodiments, the pump 16 can be an infusion pump though other fluid pumps are possible. The pump 16 can pump the fluid at a constant rate, or intermittently. The pump 16 can pump the fluid at a rate determined by an operator of the pump (e.g., a caregiver, a patient, among other possibilities). In embodiments, the fluid delivery system 1 can include a cassette that can fit into a corresponding socket of the pump 16.

The fluid delivery system 1 includes a device 100 for removing a gas (e.g., air) from the fluid flowing through the fluid delivery system 1. In embodiments, the device 100 can be a degasser, an air catch, among other possibilities. The device 100 is provided between the fluid source 10 and the fluid destination 12 to reduce and/or eliminate gas from the fluid before the fluid reaches the fluid destination 12. The device 100 can be directly fluidly connected to the pump 16 or can be fluidly connected to the pump via supply lines 14.

The device 100 can include a filter, a body defining an interior, and the filter can be within the interior and can segment the interior into a first chamber and a second chamber. The device 100 can include an inlet that can communicate the fluid into the first chamber, and a first outlet can communicate the fluid out of the first chamber. In embodiments, the device 100 can include a second outlet this configured to communicate the gas out of the second chamber. The filter can be configured such that it is impermeable to liquid to isolate the second chamber from liquid of the fluid when the fluid is in the first chamber. The filter can also be permeable to gas and can be configured to permit communication of the gas from the first chamber to the second chamber to separate the gas from the liquid of the fluid. The device 100 can configured to separate gas from the fluid without substantially impacting flow rates through the fluid delivery system 1. In embodiments, this can be achieved due to the structure of the interior of the device 100 that the fluid flows through. For example, the interior of the device 100 can be provided without any structures (e.g., filters, fluid flow disrupters, among other possibilities) that restrict flow the fluid through the device 100. In embodiments, the entirety of the flow path through the device can be at least as restrictive as the most restrictive point of the fluid delivery system. By not substantially reducing the flow rate of the fluid through the fluid delivery system, the device 100 can be used for example with pumps sized for the fluid delivery system without impacting the operation of the pumps.

The device 100 can separate the gas from the fluid passively, that is, the device 100 can be inserted into the flow path of the fluid of the fluid delivery system 1 and no external source of energy is needed to separate the gas from the fluid flowing through the fluid delivery system 1. For example, fluid can flow through the inlet of the device 100 into the first chamber. Once within the first chamber, gas contained within the fluid will naturally rise up towards the filter due to, for example, buoyancy of the gas within the fluid. When the gas reaches the filter, because the filter is permeable to gas the gas will pass through the filter and into the second chamber. Because the filter is impermeable to fluid, only the gas will pass through the filter under normal operating conditions (though if the fluid applies too much pressure to the filter it is possible that at least some fluid can break through the filter and spill into the second chamber). The fluid within the first chamber and egress out of the first chamber via first outlet. In embodiments, the gas can egress out of the second chamber via the second outlet.

In embodiments, a top of an inner surface of the body that defines a top of the interior of the body can taper towards the filter and/or towards the second chamber. This can help direct the gas towards the filter to improve gas separation efficiency of the device 100. In embodiments, the second outlet can include a bottom surface that slopes downwardly away from the second chamber. According to this configuration, fluid in an external environment surrounding the device can be directed away from the second outlet to prevent contamination of the second chamber. In embodiments, the device 100 can include projections within the second chamber that extend along the filter in the second chamber. These projections can help resist pressure applied to the filter by the fluid within the first chamber to improve longevity of the filter. In embodiments, the projections can also form sub-chambers within the second chamber. The sub-chambers can segment the second chamber. According to this configuration, fluid that enters the second chamber (via the second outlet or due to localized overpressure of the filter) can be isolated within sub-chambers to improve the efficiency of gas permeation of the filter in the other sub-chambers.

The device 100 can be provided upstream from the pump 16. That is, the device 100 can be interposed between the fluid source 10 and the pump 16. In embodiments, the device 100 can be attached to the pump 16. For example, the device 100 can be attached to the pump 16 at an inlet 18 of the pump 16. In such embodiments, the device 100 can remove gas from the fluid before the fluid enters the inlet 18 of the pump 16. This can reduce or eliminate the amount of gas flowing between the inlet 18 and an outlet 20 of the pump 16, which can for example reduce the incidence of pump failures and pump alarms, improve longevity of the pump 16, and/or reduce costly downtime of the fluid delivery system 1 caused by gas flowing through the pump 16. In embodiments, the device 100 can be removably attached to the pump 16. For example, the device 100 can include a connector that removably connects to the pump 16. In embodiments, the connector of the device 100 can removably connect directly to the inlet 18. Additionally, or alternatively, the connector of the device 100 can connect to another structure of the pump 16 such as a body of the pump 16 and/or a complimentary connector of the pump 16. The connector of the device 100 can form a friction fit, a snap fit, a threaded connection, or any other functional equivalent connection with the pump 16.

The device 100 can take the form of a number of different embodiments, such as the devices 200, 300, 400, 500, 600, 700, and 800, described later. Except such structures, features, relationships, and/or functionalities that persons of skill in the art would recognize as clearly mutually exclusive, the device 100 can include any of the structures, features, relationships, and/or functionalities of any of the devices 200, 300, 400, 500, 600, 700, and 800, described later, and vice versa.

FIG. 1B shows a schematic view of another fluid delivery system 2 according to aspects of the invention. The fluid delivery system 2 can include each of the structures, features, relationships, and functionality described in reference to the fluid delivery system 1, except the device 100 can be provided downstream from the pump 16. That is, the device 100 can be interposed between the pump 16 and the fluid destination 12. In such embodiments, the device 100 can remove gas from the fluid after the fluid has been pumped through the pump 16, which can remove gas in the fluid including gas degassed as the fluid is pumped through the pump 16. In embodiments, the device 100 can be attached to the pump 16 at the outlet 20 of the pump 16.

FIG. 1C shows a schematic view of another fluid delivery system 3 according to aspects of the invention. The fluid delivery system 3 can include each of the structures, features, relationships, and functionality described in reference to the fluid delivery systems 1, 2, except the fluid delivery system 3 can be provided without the pump 16. For example, in embodiments without the pump 16 the fluid can be driven from the fluid source 10 to the fluid destination 12 by gravity or a pressure differential (e.g., a syphon).

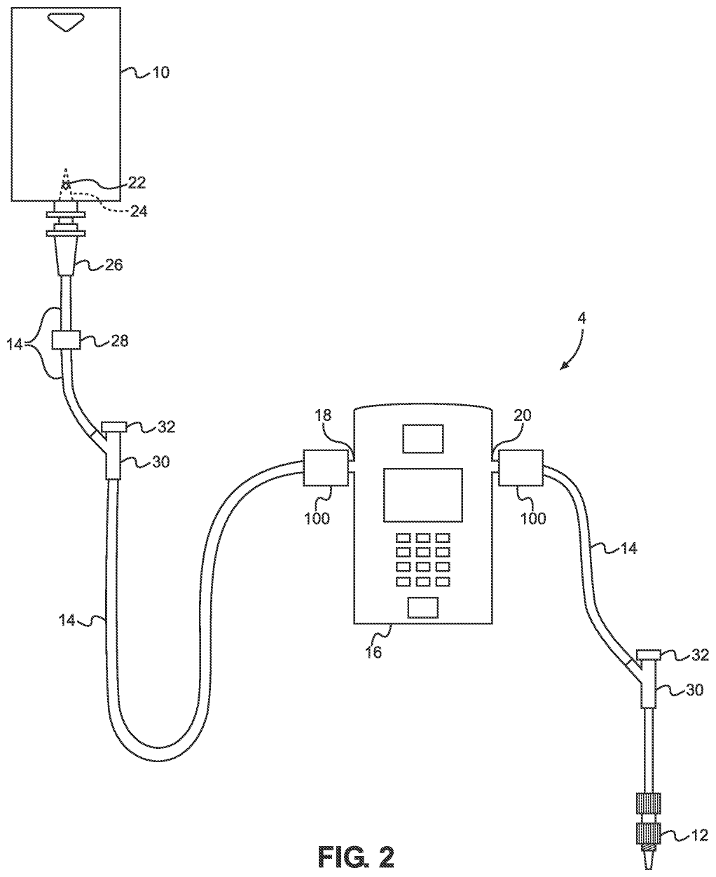

FIG. 2 shows a schematic view of another fluid delivery system 4 according to aspects of the invention. The fluid delivery system 4 can include each of the structures, features, relationships, and functionality described in reference to the fluid delivery systems 1 and/or 2 (and vice versa), and the fluid delivery system 4 can be particularly suited for delivering fluid to a patient. For example, in embodiments of the fluid delivery system 4 the fluid source 10 can be an intravenous (IV) bag, a bottle, or other closed sterile container. The fluid delivery system 4 can be used to deliver a variety of fluids to a patient (e.g., to a blood vessel of the patient) including, for example, volume expanders (e.g., a saline solution (NaCl) as fluid replacement to fight dehydration, a glucose solution etc.), whole blood (e.g., a blood transfusion), blood components (e.g., red blood cells, plasma, platelets, etc.), medicine (e.g., chemotherapy medicine, antibiotics, etc.), combinations thereof, among other possibilities.

The fluid destination 12 of the fluid delivery system 4 can be, for example, a catheter that can be inserted through the skin and into a vein or a port that can be implanted into the skin of a patient. The vein may be a peripheral vein (e.g., a vein in an arm or leg) or a central vein (e.g., a vein in the head or chest). In embodiments, the fluid delivery system 4 can deliver fluids into the body of the patient through another device, such as a port implanted in the skin of the patient. The fluid destination can include, for example, a male Luer connector that can be fixedly coupled to the supply lines 14 (e.g., fused via heat or adhesive) and that can be coupled to a corresponding female Luer connector. In embodiments, the fluid destination 12 can be coupled to the supply lines with another type of connector (e.g., via a screw-type or pressure fitting, etc.).

The device 100 can remove gas (e.g., air) bubbles from the fluid delivery system 4 before the gas bubbles reach the patient. By reducing the amount of gas introduced into the blood vessel of the patient, the likelihood of complications related to air in the blood vessels, such as an air embolus causing an embolism, is reduced. In embodiments, the fluid delivery system 4 can include two devices 100. One device 100 can be provided upstream from the pump 16 and another device 100 can be provided downstream from the pump 16.

In embodiments, the pump 16 can include a sensor to detect the presence of gas in the fluid pumped through the pump 16. In such embodiments, the fluid delivery system 4 can take precautionary measures when the sensor detects gas (e.g., a predetermined amount of gas) in the fluid. The precautionary measures can include halting the flow of fluid through the fluid delivery system 4, and/or triggering an alert (e.g., an audial alarm, a visual signal such as a flashing light, a digital signal such as message to a phone or other smart device, etc.). In embodiments in which the device 100 is positioned upstream from the pump 16 such as shown in FIG. 1A, the device 100 can reduce the occurrences of these alerts, which can reduce the disturbances of both the patient and the caregiver. In alternative embodiments such as shown in FIG. 1B, the device 100 can be positioned downstream from the pump 16.

In embodiments such as shown in FIG. 2, the fluid delivery system 4 can include multiple devices 100 including at least one device 100 positioned upstream from the pump 16 and at least one other device 100 positioned downstream from the device 100. The combination of the upstream device 100 and the downstream device 100 can capture and/or remove gas at multiple points along the fluid delivery system 4 including both upstream and downstream from the pump 16. According to this configuration, the upstream device 100 can capture and/or vent gas from the fluid before the fluid reaches the pump 16, which can reduce the occurrences of pump alarms and down time associated with addressing such alarms. Further, the downstream device 100 can capture and/or vent gas (e.g., champagne bubbles) released from the fluid when the fluid passes through the pump 16, which can mitigate or eliminate migration of such released gases back into the pump 16 and thereby further reduce the occurrences of pump alarms and down time associated with addressing such alarms.

In embodiments such as shown in FIG. 2, the fluid source 10 can include an opening 22. The fluid delivery system 4 can include a spike 24 that can be connected to the supply lines 14 and that can pierce the opening 22 to permit fluid to flow from the fluid source 10 into the supply lines 14. The fluid delivery system 4 can include a drip chamber 26 that can be coupled to the spike 24 and the supply lines 14. The fluid can drip or otherwise flow out of the fluid source 10, through the opening 22, and into the drip chamber 26 at a controlled rate. The size of the opening 22 can be configured to achieve a desired drop size and rate. When the fluid delivery system 4 is primed (i.e., generally filled with fluid) and in use, the drip chamber 26 can generally only partially fill with fluid. The fluid can pass from the drip chamber 26 to the remainder of the fluid delivery system 4 (e.g., via the supply lines 14) while much of the gas in the system remains in the drip chamber 26 or flows back into the fluid source 10 through the opening 22. However, some gas may pass with the fluid from the drip chamber 26 to the remainder of the fluid delivery system 4 and the device 100 can remove some or all of such gas from the fluid.

From the drip chamber 26, the fluid can pass via the supply lines 14 through other components of the fluid delivery system 4, such as a check valve 28 (or clamp) and one more connectors 30. The connectors 30 can be forked tubes or y-site. The fluid delivery system 4 can be joined or piggybacked to another fluid delivery system (e.g., a secondary set, piggyback set, etc.) via the connector 30. The connector 30 can include a junction with a port 32 (e.g., med port, injection port, etc.), which can allow another substance to be introduced into the fluid delivery system 4. The other substance can be, for example, a second fluid from a second fluid source. The second fluid can be delivered from a fluid source similar to the fluid source 10 via tubing that interfaces with the connector 30, e.g., from a syringe with a needle that pierces the port 32 or from a syringe that interfaces with the connector 30 in another way (e.g., a Luer connector or another needleless connector). Introduction of a second fluid into the fluid delivery system 1 may introduce additional gas into the system, which can be reduced or eliminated from the fluid via the device 100 if provided the device 100 is located downstream from the connector 30.

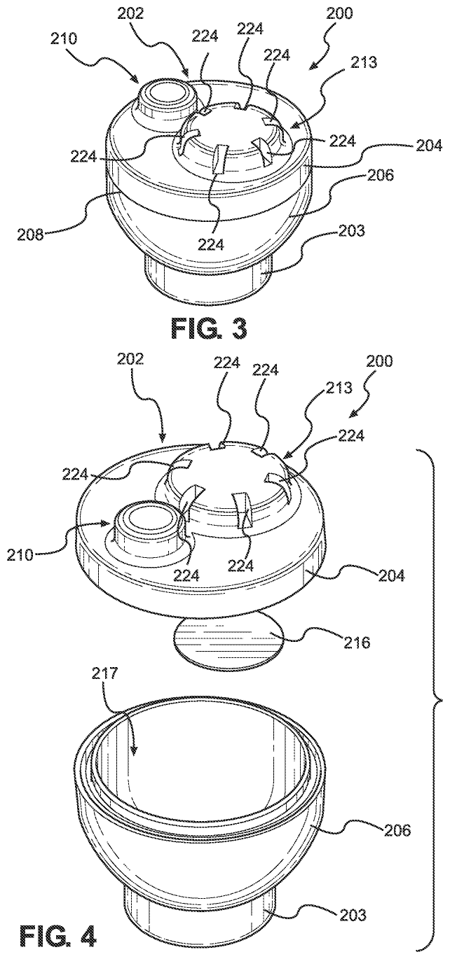

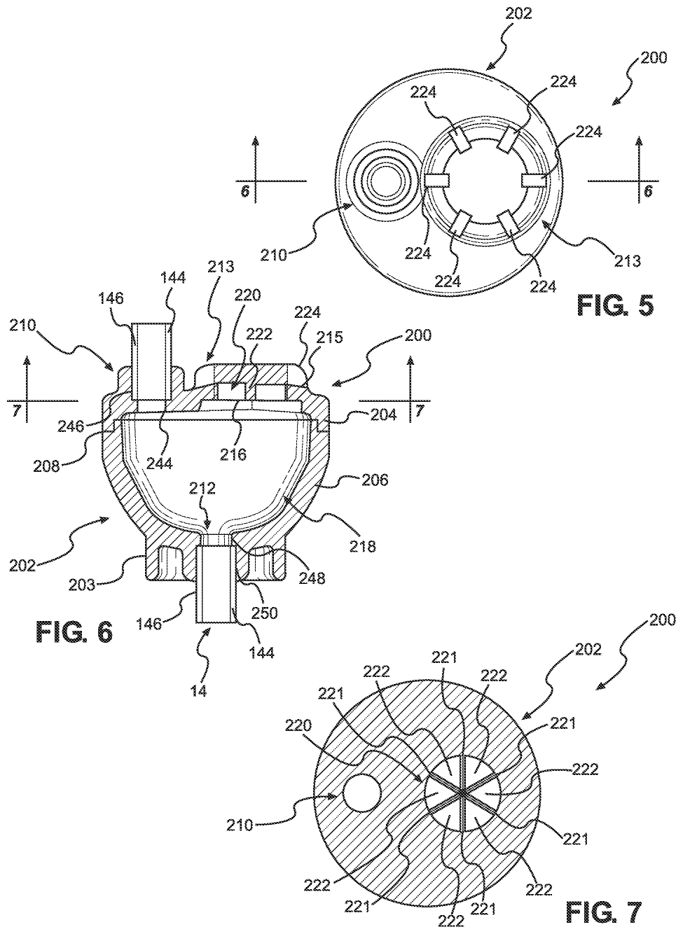

FIGS. 3-7 show views of a device 200 for removing a gas (e.g., air) from a fluid according to aspects of the invention. FIG. 3 shows a perspective view of the device 200. FIG. 4 shows an exploded view of the device 200. FIG. 5 shows a top view of the device 200. FIG. 6 shows a cross-section view along section line 6-6 of the device 200 shown in FIG. 5. FIG. 7 shows a cross-section view along section line 7-7 of the device 200 shown in FIG. 6. The device 200 is an embodiment of the device 100, previously described. Except such structures, features, relationships, and/or functionalities that persons of skill in the art would recognize as clearly mutually exclusive, the device 200 can include any of the structures, features, relationships, and/or functionalities of the device 100 and vice versa. The device 200 can be used with any or all of the fluid delivery systems 1, 2, 3, or 4 to remove gas from a fluid flowing through any of the respective fluid delivery systems 1, 2, 3, or 4.

The device 200 can include a body 202. The body 202 can be formed of a material that can be sterilized with high-energy radiation, that is bio compatible, that is chemical resistant, and/or that is transparent. For example, the body 202 can be formed of a plastic such as a polycarbonate. In embodiments, the body 202 can be an integral body. The term “integral” as used herein can include the plain and ordinary meaning and can mean a unitary part that can, for example, be molded or joined in a permanent manner during a manufacturing process. Alternatively, at least some of the body 202 can be formed as distinct portions that are subsequently fixedly or removably joined together. For example, in embodiments the body 202 can include a first portion 204 (e.g., a top), which can be arranged at a top of the device 200, and a second portion 206 (e.g., a bottom), which can be arranged at a bottom of the device 200. In embodiments, one or more inner surface of the first portion 204 and/or the second portion 206 can taper towards respective outlets 212, 213 (described later) to direct liquid and/or or gas towards the respective outlets 212, 213. In embodiments in which the body 202 is segmented into portions, the portions (e.g., the first portion 204 and the second portion 206) can be joined together at one or more interface 208 via any number of known techniques including with welds (e.g., with a sonic weld), fasteners (e.g., a screw, bolt, connector, etc.), adhesive, among other possibilities. In embodiment, the body 202 can include a base 203 that can project downwardly from a bottom of the body 202. The base 203 can support the device 200. For example, the base 203 can support the device 200 on top of the pump 16.

The device 200 can include an inlet 210 that can receive a fluid. In embodiments in which the device 200 is provided upstream from the pump 16, the inlet 210 can be operatively (e.g., directly or indirectly) fluidly connected to the supply lines 14 upstream from the pump 16 to receive the fluid therefrom. In embodiments in which the device 200 is provided downstream from the pump 16, the inlet 210 can be operatively (e.g., directly or indirectly via supply line 14) fluidly connected to the outlet 20 of the pump 16 to receive the fluid therefrom. The inlet 210 can project (e.g., upwardly) from the body 202. In embodiments, the inlet 210 can be formed integrally with the body 202. Alternatively, the inlet 210 can be a separate component from the body 202 joined to the body 202 via any number of known techniques.

The device 200 can include a fluid outlet 212 (e.g., a first outlet) that can expel the fluid. In embodiments in which the device 200 is provided upstream from the pump 16, the fluid outlet 212 can be operatively (e.g., directly or indirectly via supply lines 14) fluidly connected to the inlet 18 of the pump 16 and can expel the fluid from the device 200 to the pump 16. In embodiments in which the device 200 is provided downstream from the pump 16, the fluid outlet 212 can be operatively (e.g., directly or indirectly) fluidly connected to supply lines 14 downstream from the pump 16 to expel the fluid thereto. The fluid outlet 212 can project (e.g., downwardly) from the body 202. In embodiments, the fluid outlet 212 can be formed integrally with the body 202. Alternatively, the fluid outlet 212 can be a separate component from the body 202 joined to the body 202 via any number of known techniques.

The device 200 can include a gas outlet 213 (e.g., a second outlet) that can expel gas from the device 200 from the interior 217 of the body. The gas outlet 213 can project (e.g., upwardly) from the body 202. The gas outlet 213 can be arranged at a top of the device 200, which leverage the naturally tendency of gas to rise in fluids to improve separation of the gas from the fluid.

In embodiments, the gas outlet 213 can be formed integrally with the body 202. Alternatively, the fluid outlet 212 can be a separate component from the body 202 joined to the body 202 via any number of known techniques. In embodiments, the gas outlet 213 can be larger than the fluid outlet 212, which can increase the capacity of the gas removal for the device 200. In embodiments, a center of each the fluid outlet 212 and the gas outlet 213 can be laterally offset from a center of the top of the body 202, which can maximize the size of the gas outlet 213 and improve the gas removal capacity of the device 200. The gas outlet 213 can include one or more bottom surface 215 that slopes downwardly away from the second chamber 220 (described later). According to this configuration, liquid in an external environment surrounding the device 200 can be at least partially directed away from the second chamber 220 to reduce contamination of the second chamber 220 and improve the efficacy of the filter 216 (described later). The gas outlet 213 can include vents 224. The vents 224 can be disposed through the body 202 to fluidly connect the second chamber 220 with an external environment surrounding the device 200. According to this configuration, gas removed from the fluid through the filter 216 can be vented from the second chamber 220 to the external environment. This can free up space within the second chamber 220 and allow for further gas removal from the fluid.

In embodiments such as shown in FIGS. 3-7, the inlet 210 and the gas outlet 213 can at the top of the device 200 and the fluid outlet 212 can be at the bottom of the device. This arrangement can be advantageous for example for connecting to pumps that have an inlet arranged at the top of the device.

The device 200 can include a filter 216. The filter 216 can segment an interior 217 of the body 202 into chambers, for example, a first chamber 218 and a second chamber 220. The inlet 210 can be fluidly connected (e.g., can open into) the first chamber 218 and the fluid outlet 212 can be fluidly connected to (e.g., can open out of) the first chamber 218. The filter 216 can be wholly or substantially impermeable to liquid (e.g., water) in the fluid flowing through the second chamber 220 from the inlet 210 and through the fluid outlet 212. Put differently, the filter 216 can wholly or substantially isolate the second chamber 220 from the liquid components of the fluid and wholly or substantially prevent liquid communication between the first chamber 218 and the second chamber 220. The filter 216 can be permeable to gas (e.g., air) contained within the fluid flowing through the first chamber 218 and can be configured such that gas contained within the fluid can be communicated from the first chamber 218 to the second chamber 220. According to this configuration, gas contained within the fluid can be removed from the fluid and isolated from the fluid within the second chamber 220 by permeating though the filter 216 and moving to the second chamber 220. Accordingly, the device 200 can remove gas from the fluid as the fluid passes through the device 200.

The filter 216 can be formed of a material that is impermeable to a fluid (e.g., water) flowing through device 200 and that is permeable to a gas (e.g., air) contained within the fluid. In embodiments, the filter 216 can be a membrane. In embodiments, the filter 216 can be formed of a membrane with pores. In embodiments, the pores can have a diameter of 0.2 microns (in embodiments, +/−10%, +/−5%, +/−2.5%, among other possibilities). The filter 216 can be formed of a hydrophobic material. In embodiments, the material that forms the filter 216 can also be an oleophobic material. In embodiments, the filter 216 can be formed of a material that can resist degradation when exposed to caustic agents, such as for example medicines used in chemotherapy treatments. The device 200 integrated with the filter 216 can thus be advantageous for use in safely delivering chemotherapy treatments. The filter 216 can be formed of PTFE, an acrylic copolymer matrix, non-woven nylon, combinations thereof, or other materials. The filter 216 can have a thickness of between 0.15 and 0.30 mm. The filter 216 can have a water entry pressure greater than or equal to 750 mbar, greater than or equal to 1000 mbar, or greater than or equal to 1790 mbar. The filter 216 can have a water entry pressure between 750 mbar and 1790 mbar. The filter 216 can permit air flow of at least 2 l/hr/cm² Δp 70 mbar and in embodiments at least 5 l/hr/cm² Δp 70 mbar. The filter 216 can permit air flow of between 2 l/hr/cm² Δp 70 mbar and 5 l/hr/cm² Δp 70 mbar. The filter 216 can permit air flow of 4.2 SLPM (in embodiments, +/−10%, +/−5%, +/−2.5%, among other possibilities). In embodiments, the filter 216 can be disk shaped to improve manufacturability of the filter 216, though other shapes (e.g., rectangular shapes) are possible.

In embodiments, the filter 216 can be held within the interior 217 of the body 202 in a manner that prevents fluid communication between the first chamber 218 and the second chamber 220. For example, the filter 216 can be sandwiched within the interface 208 between the first portion 204 and the second portion 206. In embodiments, the filter 216 can be sealed to interior walls of the body 202 with an adhesive or a weld (e.g., a sonic weld), though other techniques of attaching the filter 216 to the interior of the body 202 are possible.

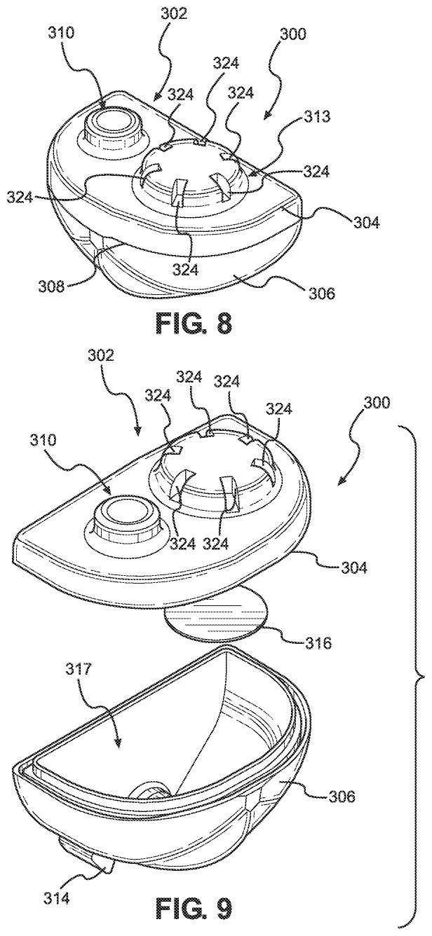

In embodiments, the body 202 can include projections 222 that project into the second chamber 220. The projections 222 can resist pressure exerted by the fluid within the first chamber 218 on the filter 216, which can reduce fatigue of the filter 216 and prolong the life of the filter 216. In embodiments, the projections 222 can project from an inner wall of the first portion 204 inwardly towards the second chamber 220. In embodiments, the projections 222 can project along a surface of the filter 216 (i.e., the surface of the filter that faces the second chamber 220). In embodiments such as shown in FIG. 7, the projections 222 can project radially inwardly into the second chamber 220 and intersect at a central portion of the second chamber 220 such that the projections 222 define a star-shaped pattern. In embodiments such as shown in FIG. 8, the projections can extend perpendicular (i.e., upwardly from the surface of the filter 216 towards the gas outlet 213) to the filter and segment the second chamber into a plurality of sub-chambers 221. In embodiments, each of the sub-chambers 221 can respectively fluidly communicate with one or more of the vents 224. In embodiments, each of the sub-chambers 221 can be fluidly connected to only one respective vent 224 such that the sub-chambers 221 are completely isolated from each other within the second chamber 220. This can help isolate (from each other) regions of the surface of the filter 216 within the second chamber 220, which can help the device function to remove gas even if portions of the filter 216 are compromised with fluid that breaks through the filter and/or that enters the gas outlet 213 from an external environment. In embodiments, the projections 222 can terminate a position level with the interface 208. In embodiments in which the filter 216 is held by the interface 208 or fixed at a level of the interface 208 the terminal ends of the projections 222 can support the filter 216 at the level position to minimize fatigue and maximize longevity.

The device 200 can be configured such that, when connected to a fluid delivery system (e.g., any of the fluid delivery systems 1, 2, 3) and when primed the device 200 does not substantially reduce the flow rate of the fluid flowing through the fluid delivery system. In embodiments, the device 200 can be configured such that, both when primed and when not primed, the device 200 does not substantially reduce the flow rate of the fluid flowing through the fluid delivery system. For example, the device 200 can be configured such that no portion of a flow path for the fluid defined by the device 200 (e.g., through the inlet 210, the first chamber 218, the fluid outlet 212) is smaller than the smallest portion of the flow path for the entire fluid delivery system. In embodiments, the device 200 can be configured such that no portion of the flow path for the fluid defined by the device 200 is smaller than a flow path defined by the interior of the supply lines 14.

For example, in embodiments an inner diameter 244 of the smallest portion of the inlet 210 (i.e., a first inner diameter) can be greater than or equal to an inner diameter 144 of a supply line 14 (i.e., an inlet supply line) that connects to the inlet 210. Accordingly, the inlet 210 does not restrict flow from the supply line 14. In embodiments, an inner diameter 246 of the largest portion of the inlet 210 (i.e., a second inner diameter) can be greater than or equal to an outer diameter 146 of the supply line 14 (i.e., the inlet supply line). According to this configuration, the supply line 14 can be inserted into the inlet 210 without crimping the interior of the supply line 14 and without restricting the flow path. The inner diameter 244 can be between the inner diameter 246 and the interior 217 of the body 202. The inner diameter 246 can be greater than the inner diameter 244 and the inlet 210 can include a continuous or discontinuous step from the inner diameter 246 to the inner diameter 244. According to this configuration, the step from the inner diameter 246 to the inner diameter 244 can be dimensioned to prevent over insertion of the supply line 14 into the device 200.

In embodiments, an inner diameter 248 of the smallest portion of the fluid outlet 212 (i.e., a first inner diameter) can be greater than or equal to an inner diameter 144 of a supply line 14 (i.e., an outlet supply line) that connects to the fluid outlet 212. Accordingly, the fluid outlet 212 does not restrict flow from the supply line 14.

In alternative embodiments (not shown), the inner diameter 248 can be smaller than the inner diameter 144 of the supply line (i.e., the outlet supply line), but larger enough that a cross-sectional flow area defined by the fluid outlet 212 at the inner diameter 248 is not the smallest cross-sectional flow area of the flow path if the fluid delivery system. For example, the smallest cross sectional flow area (i.e., a first cross-section flow area) of the fluid delivery system can occur for example at an outlet of a needle at the fluid destination or at some other point along the flow path. The supply line 14 (i.e., the outlet supply line) can define a cross-sectional flow area (i.e., a second cross-sectional flow area) through which the fluid can flow and the fluid outlet 212 at the inner diameter 248 can define a cross-sectional flow area (i.e., a third cross-sectional flow area) through which the fluid can flow. The third cross-sectional flow area can be greater than the first cross-sectional flow area and the third cross-sectional flow area can be less than the second cross-sectional flow area. This configuration can be advantageous to prime the device 200 (e.g., when the device 200 is disposed downstream from the pump 16) without restricting flow through the fluid delivery system when the device 200 is primed since the fluid outlet 212 is not the narrowest part of the flow path.

In embodiments, an inner diameter 250 of the largest portion of the fluid outlet 212 (i.e., a second inner diameter) can be greater than or equal to an outer diameter 146 of the supply line 14 (i.e., the outlet supply line). According to this configuration, the supply line 14 can be inserted into the fluid outlet 212 without crimping the interior of the supply line 14 and without restricting the flow path. The inner diameter 248 can be between the inner diameter 250 and the interior 217 of the body 202. The inner diameter 250 can be greater than the inner diameter 248 and the fluid outlet 212 can include a continuous or discontinuous step from the inner diameter 250 to the inner diameter 248. According to this configuration, the step from the inner diameter 250 to the inner diameter 248 can be dimensioned to prevent over insertion of the supply line 14 into the device 200.

In embodiments, the device 200 can continuously remove gas from the fluid through the vents 224. In embodiments, the device 200 can hold between 1 mL and 1.8 mL of gas, though the device 200 can remove more gas by venting the gas through the vents 224. In embodiments, the device 200 can function to remove gas from the fluid when rotated. For example, the device 200 can function to remove gas from the fluid when rotated up to 90 degrees clockwise or counterclockwise relative to the orientation shown in FIG. 7.

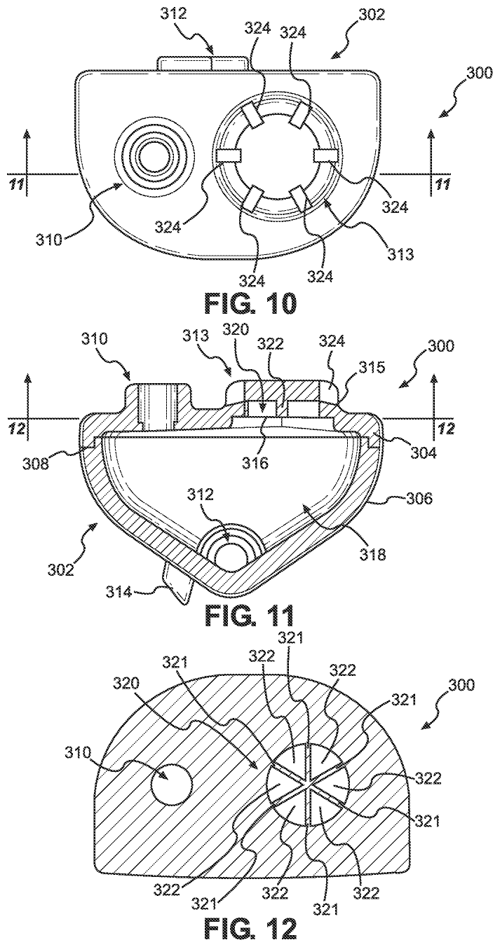

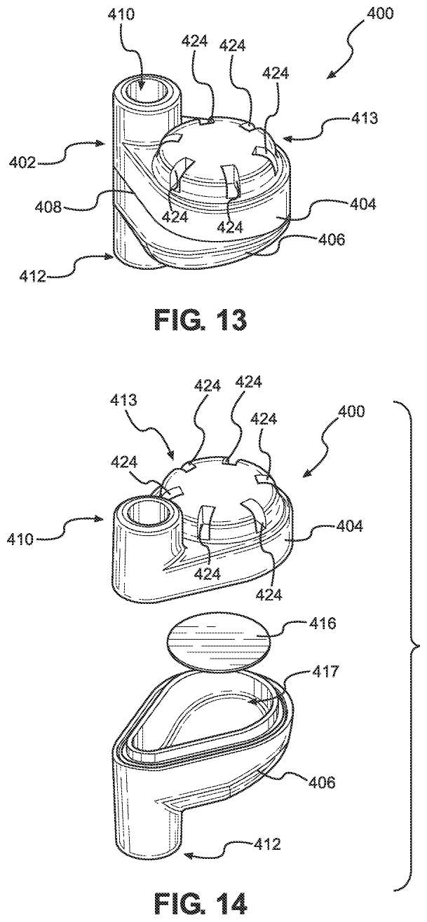

FIGS. 8-12 show views of a second embodiment of the device 300 for removing a gas (e.g., air) from a fluid according to aspects of the invention. FIG. 8 shows a perspective view of the device 300. FIG. 9 shows an exploded view of the device 300. FIG. 10 shows a top view of the device 300. FIG. 11 shows a cross-section view along section line 11-11 of the device 300 shown in FIG. 10. FIG. 12 shows a cross-section view along section line 12-12 of the device 300 shown in FIG. 11. The device 300 is an embodiment of the device 100, previously described. Except such structures, features, relationships, and/or functionalities that persons of skill in the art would recognize as clearly mutually exclusive, the device 300 can include any of the structures, features, relationships, and/or functionalities of the devices 100, 200, and vice versa. For example and as shown in FIGS. 8-12, the device 300 can include any or all of a body 302, a first portion 304, a second portion 306, an interface 308, an inlet 310, a fluid outlet 312, a gas outlet 313, a filter 316, a bottom surface 315, an interior 317, a first chamber 318, a second chamber 320, sub-chambers 321, projections 322, and vents 324, which can include any of the features, relationships, and/or functionalities described previously with respect to like structures of the device 200. The device 300 can be used with any or all of the fluid delivery systems 1, 2, 3, or 4 to remove gas from a fluid flowing through any of the respective fluid delivery systems 1, 2, 3, or 4.

In embodiments, the device 300 can be configured to interface horizontally with a pump, which can be advantageous for pumps having inlets and/or outlet on a side of the pump. For example, the device 300 can include a protrusion 314 that can protrude from the device 300. In embodiments, the protrusion 314 can interface with the pump 16 to, for example, stabilize the device 300 by resisting a force generated from the weight of the device 300 that would tend to pull the device 300 out of the pump 16. In embodiments, the protrusion 314 can be a connector, which can connect the device 300 to the pump 16. For example, the protrusion 314 can define a shape the is complementary to a shape of a portion of the pump 16 and that can fit to the shape of the portion of the pump 16 to directly connect the device 300 to the pump 16. The connection between the protrusion 314 and the pump 16 can be, for example, a snap fit connection, a press-fit connection, or other similar type of connection. In embodiments, the protrusion 314 can be integral with the body 302, the inlet 310, and/or the fluid outlet 312. For example, in embodiments such as shown in FIGS. 8-12 the protrusion 314 can be integral with the body 302 and the fluid outlet 312. Alternatively, the protrusion 314 can be a distinct component joined to the body 302 via any number of known techniques.

The fluid outlet 312 of the device 300 can extend from a side of the body 302. For example, the fluid outlet 312 can extend from a side of the body 302 perpendicular to the inlet 310 and the gas outlet 313. According to this configuration, the device 300 can easily connect to a pump having an inlet/outlet on the side of the pump.

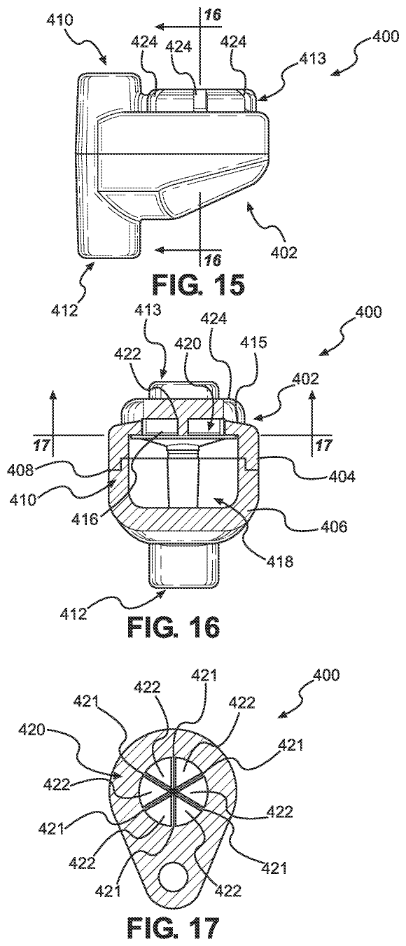

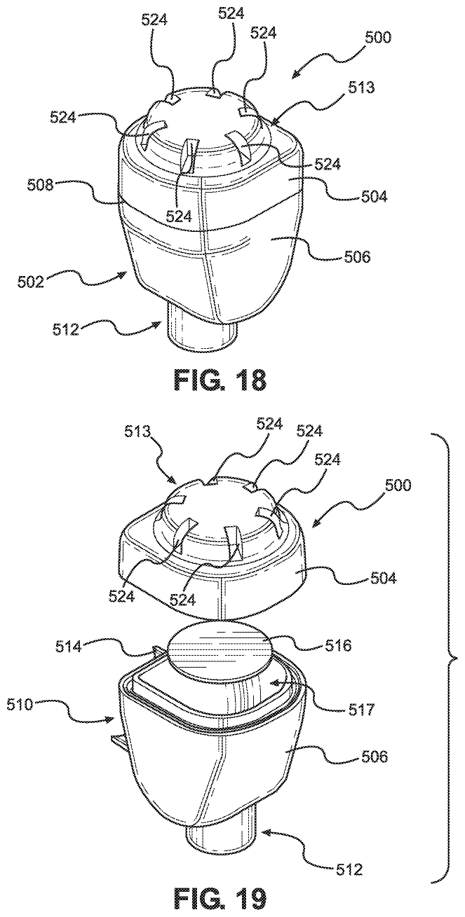

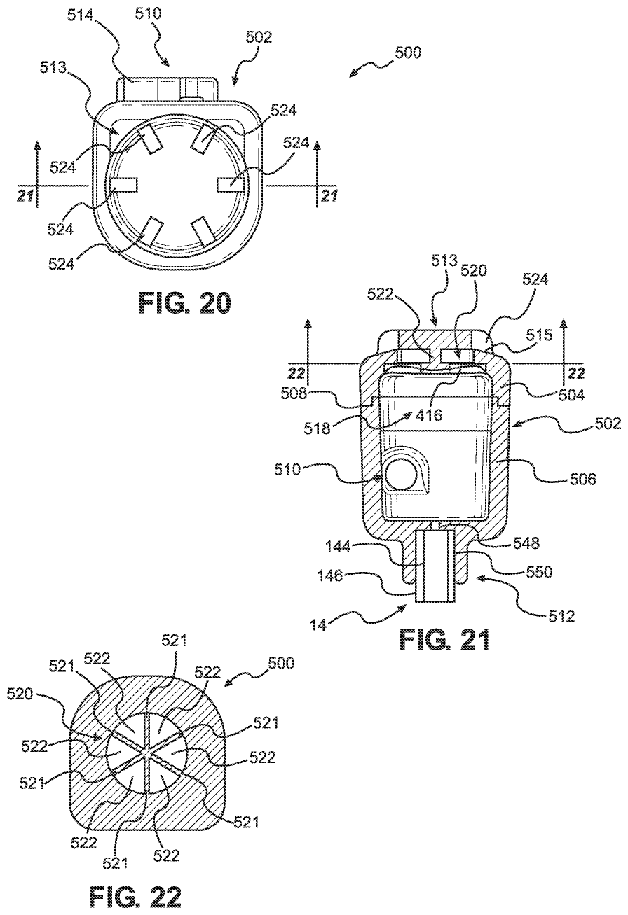

FIGS. 15-19 show views of a third embodiment of the device 400 for removing a gas (e.g., air) from a fluid according to aspects of the invention. FIG. 18 shows a perspective view of the device 400. FIG. 19 shows an exploded view of the device 400. FIG. 20 shows a top view of the device 400. FIG. 21 shows a cross-section view along section line 21-21 of the device 400 shown in FIG. 20. FIG. 22 shows a cross-section view along section line 22-22 of the device 400 shown in FIG. 21. Except such structures, features, relationships, and/or functionalities that persons of skill in the art would recognize as clearly mutually exclusive, the device 400 can include any of the structures, features, relationships, and/or functionalities of the devices 100, 200, 300, and vice versa. For example and as shown in FIGS. 18-22, the device 400 can include any or all of a body 402, a first portion 404, a second portion 406, an interface 408, an inlet 410, a fluid outlet 412, a gas outlet 413, a filter 416, a bottom surface 415, an interior 417, a first chamber 418, a second chamber 420, sub-chambers 421, projections 422, vents 424, which can include any of the features, relationships, and/or functionalities described previously with respect to like structures of the devices 200 and/or 400. The device 400 can be used with any or all of the fluid delivery systems 1, 2, 3, or 4 to remove gas from a fluid flowing through any of the respective fluid delivery systems 1, 2, 3, or 4.

The device 400 can be configured to function downstream from a pump. For example, the device 400 can have a form factor that it smaller than a form factor of a similar device configured to function upstream from the pump. The device 400 can be configured to interface horizontally with the pump. The inlet 410 and the fluid outlet 412 can be concentrically aligned and the body 402 can protrude offset laterally from the concentrically aligned inlet 410 and fluid outlet 412.

FIGS. 18-22 show views of a fourth embodiment of the device 500 for removing a gas (e.g., air) from a fluid according to aspects of the invention. FIG. 18 shows a perspective view of the device 500. FIG. 19 shows an exploded view of the device 500. FIG. 20 shows a top view of the device 500. FIG. 21 shows a cross-section view along section line 21-21 of the device 500 shown in FIG. 20. FIG. 22 shows a cross-section view along section line 22-22 of the device 500 shown in FIG. 21. Except such structures, features, relationships, and/or functionalities that persons of skill in the art would recognize as clearly mutually exclusive, the device 500 can include any of the structures, features, relationships, and/or functionalities of the devices 100, 200, 300, 400, and vice versa. For example and as shown in FIGS. 18-22, the device 500 can include any or all of a body 502, a first portion 504, a second portion 506, an interface 508, an inlet 510, a fluid outlet 512, a gas outlet 513, a protrusion 514, a filter 516, a bottom surface 515, an interior 517, a first chamber 518, a second chamber 520, sub-chambers 521, projections 522, vents 524, inner diameter 548 of the fluid outlet 512, and inner diameter 550 of the fluid outlet 512, which can include any of the features, relationships, and/or functionalities described previously with respect to like structures of the devices 200, 300 and/or 400. The device 500 can be used with any or all of the fluid delivery systems 1, 2, 3, or 4 to remove gas from a fluid flowing through any of the respective fluid delivery systems 1, 2, 3, or 4.

The device 500 can be configured to function downstream from a pump. For example, the device 500 can have a form factor that it smaller than a form factor of a similar device configured to function upstream from the pump. As previously described, the inner diameter 548 can be smaller than the inner diameter 144 of the supply line (i.e., the outlet supply line), but larger enough that a cross-sectional flow area defined by the fluid outlet 512 at the inner diameter 548 is not the smallest cross-sectional flow area of the flow path if the fluid delivery system. For example, the smallest cross sectional flow area (i.e., a first cross-section flow area) of the fluid delivery system can occur at an outlet of a needle at the fluid destination or at some other point along the flow path. The supply line 14 (i.e., the outlet supply line) can define a cross-sectional flow area (i.e., a second cross-sectional flow area) through which the fluid can flow and the fluid outlet 512 at the inner diameter 548 can define a cross-sectional flow area (i.e., a third cross-sectional flow area) through which the fluid can flow. The third cross-sectional flow area can be greater than the first cross-sectional flow area and the third cross-sectional flow area can be less than the second cross-sectional flow area. This configuration can be advantageous by generating back pressure at the fluid outlet 512 to prime the device 500 (e.g., when the device 500 is disposed downstream from the pump 16) without restricting flow through the fluid delivery system when the device 500 is primed since the fluid outlet 512 is not the narrowest part of the flow path. In embodiments, the fluid outlet 412 of the previously described device 400 can include each of the structures, features, and relationships of the fluid outlet 512 previously described.

In embodiments, the device 500 can be configured to interface horizontally with a pump, which can be advantageous for pumps having inlets and/or outlet on a side of the pump. For example, the device 500 can include the protrusion 514, as previously described. For example, in embodiments such as shown in FIGS. 18-22 the protrusion 514 can be integral with the body 502 and the inlet 510. Alternatively, the protrusion 514 can be a distinct component joined to the body 502 via any number of known techniques.

The inlet 510 of the device 500 can extend from a side of the body 502. For example, the inlet 510 can extend from a side of the body 502 perpendicular to the fluid outlet 512 and the gas outlet 513. According to this configuration, the device 300 can easily connect to a pump having an inlet/outlet on the side of the pump.

In embodiments, the inlet 510 can be arranged closer to a bottom of the device 500 than to a top of the device 500.

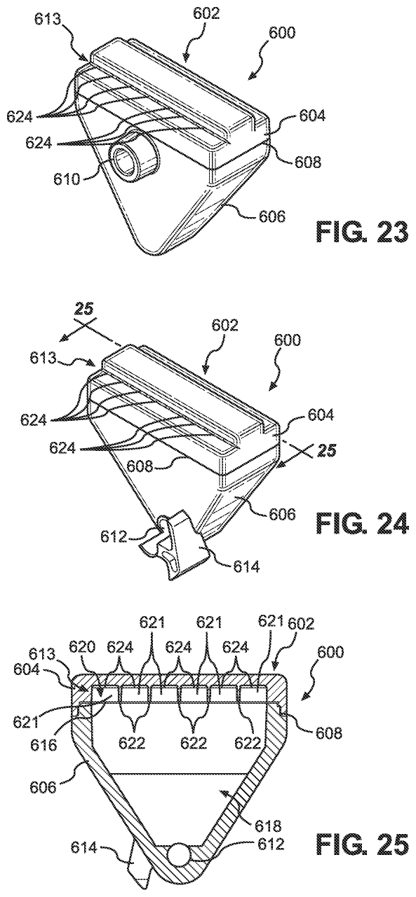

FIGS. 23-25 show views of a fifth embodiment of the device 600 for removing a gas (e.g., air) from a fluid according to aspects of the invention. FIG. 23 shows a perspective view from an inlet-side of the device 600. FIG. 24 shows a perspective view from an outlet-side of the device 600. FIG. 25 shows a cross-section view along section line 25-25 of the device 600 shown in FIG. 24. Except such structures, features, relationships, and/or functionalities that persons of skill in the art would recognize as clearly mutually exclusive, the device 600 can include any of the structures, features, relationships, and/or functionalities of the devices 100, 200, 300, 400, 500 and vice versa. For example and as shown in FIGS. 23-25, the device 600 can include any or all of a body 602, a first portion 604, a second portion 606, an interface 608, an inlet 610, a fluid outlet 612, a gas outlet 613, a protrusion 614, a filter 616, a first chamber 618, a second chamber 620, sub-chambers 621, projections 622, and vents 624, which can include any of the features, relationships, and/or functionalities described previously with respect to like structures of the devices 200, 300, 400 and/or 500. The device 600 can be used with any or all of the fluid delivery systems 1, 2, 3, or 4 to remove gas from a fluid flowing through any of the respective fluid delivery systems 1, 2, 3, or 4.

In embodiments, both the inlet 610 and the fluid outlet 612 of the device 600 can extend form a side of the body 602. For example, the inlet 610 and the fluid outlet 612 can extend from the body 602 perpendicular to the gas outlet 613. In embodiments, the gas outlet 613 can extend from a top of the body 602. In embodiments, the gas outlet 613 can extend along an entire width of the top of the body 602, which can maximize the size of the second chamber 620 and improve gas removal capacity of the device 600.

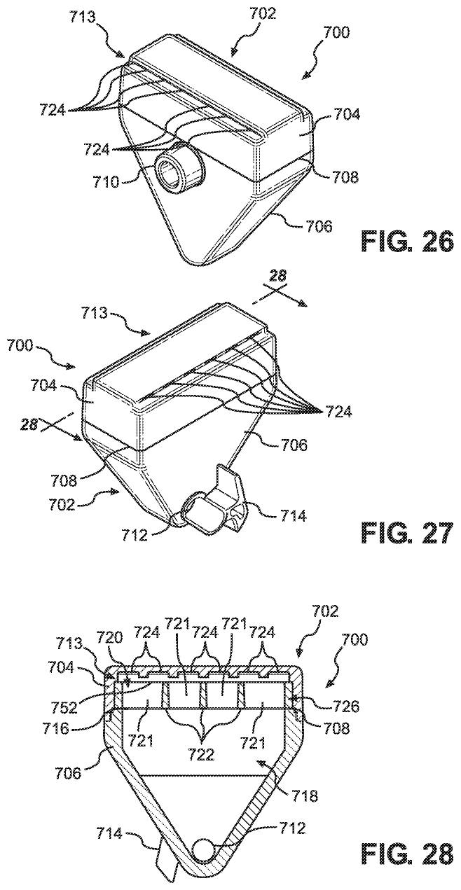

FIGS. 26-28 show views of a sixth embodiment of the device 700 for removing a gas (e.g., air) from a fluid according to aspects of the invention. FIG. 26 shows a perspective view from an inlet-side of the device 700. FIG. 27 shows a perspective view from an outlet-side of the device 700. FIG. 28 shows a cross-section view along section line 28-28 of the device 700 shown in FIG. 27. Except such structures, features, relationships, and/or functionalities that persons of skill in the art would recognize as clearly mutually exclusive, the device 700 can include any of the structures, features, relationships, and/or functionalities of the devices 100, 200, 300, 400, 500, 600 and vice versa. For example and as shown in FIGS. 26-28, the device 700 can include any or all of a body 702, a first portion 704, a second portion 706, an interface 708, an inlet 710, a fluid outlet 712, a gas outlet 713, a protrusion 714, a filter 716, a first chamber 718, a second chamber 720, sub-chambers 721, projections 722, and vents 724, which can include any of the features, relationships, and/or functionalities described previously with respect to like structures of the devices 200, 300, 400, 500 and/or 600. The device 800 can be used with any or all of the fluid delivery systems 1, 2, 3, or 4 to remove gas from a fluid flowing through any of the respective fluid delivery systems 1, 2, 3, or 4.

In embodiments, the device 700 can include a second filter 752, which can include any or all of the features, relationship, and/or functionalities of the filter 716 (i.e., the first filter). In embodiments, the second filter 752 can be arranged above the filter 716 between the vents 724 and the filter 716. In embodiments, the second filter 752 can be formed of the same material as the filter 716.

In embodiments, the second filter 752 can be different form the filter 716 in one or more respects. For example, the second filter 752 can have a pore size that is greater than a pore size of the filter 716. This arrangement can be advantageous because most or all of the liquid can be filtered out by the filter 716 with the smaller pore size while the second filter 752 can capture breakthrough fluid while also allowing the gas to pass more freely than the filter 716. In embodiments, the device 700 can mitigate against fluid breakthrough past the filter 716 due to the inclusion of the second filter 752. In embodiments, inclusion of the second filter 752 can reduce the risk of contamination of the fluid from the outside environment via the vents 724 by providing an additional barrier between outside environment and the fluid within the first chamber 718. In embodiments, the filter 716 can reduce or prevent degradation of the second filter 752 from, for example, certain caustic medicines such as chemotherapy drugs.

In embodiments, the device 700 can include a cartridge 726. The cartridge 726 can be a frame that supports the filter 716 and the second filter 752 within the body 702. In embodiments, the cartridge 726 can be mounted, adhered, welded (e.g., via a sonic weld) to the interior of the body 702, or the cartridge 726 can be sandwiched between the first portion 704 and the second portion 706. In embodiments, the cartridge 726 can include the projections 722 that define the sub-chambers 721.

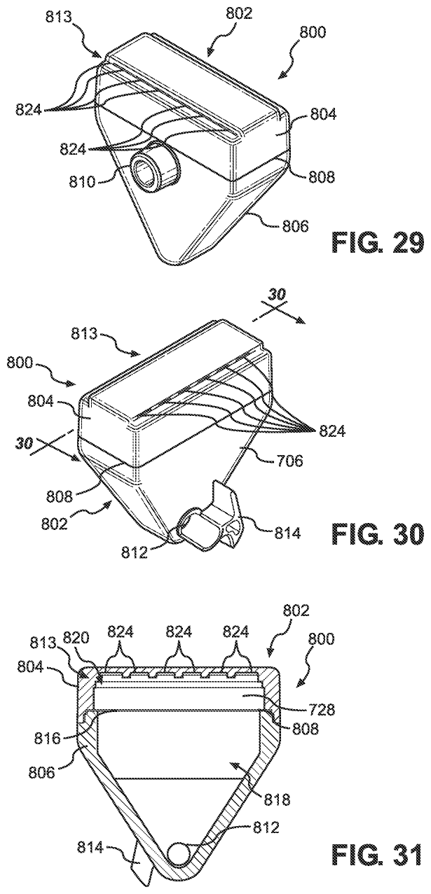

FIGS. 29-31 show views of a seventh embodiment of the device 800 for removing a gas (e.g., air) from a fluid according to aspects of the invention. FIG. 29 shows a perspective view from an inlet-side of the device 800. FIG. 30 shows a perspective view from an outlet-side of the device 800. FIG. 31 shows a cross-section view along section line 31-31 of the device 800 shown in FIG. 30. Except such structures, features, relationships, and/or functionalities that persons of skill in the art would recognize as clearly mutually exclusive, the device 800 can include any of the structures, features, relationships, and/or functionalities of the devices 100, 200, 300, 400, 500, 600, 700 and vice versa. For example and as shown in FIGS. 29-31, the device 800 can include any or all of a body 802, a first portion 804, a second portion 806, an interface 808, an inlet 810, a fluid outlet 812, a gas outlet 813, a protrusion 814, a filter 816, a first chamber 818, a second chamber 820, and vents 824, which can include any of the features, relationships, and/or functionalities described previously with respect to like structures of the devices 200, 300, 400, 500, 600 and/or 700. The device 800 can be used with any or all of the fluid delivery systems 1, 2, 3, or 4 to remove gas from a fluid flowing through any of the respective fluid delivery systems 1, 2, 3, or 4.

In embodiments, the device 800 can include a pad 828. The pad 828 can be formed of a material that absorbs liquid (e.g., water) and that is permeable to gas (e.g., air). In embodiments, the pad 828 can be arranged within the second chamber 820 above the filter 816 between the vents 824 and the filter 816. This arrangement can be advantageous because while most or all of the liquid can be filtered out by the filter 816, the pad 828 can capture breakthrough fluid while also allowing the gas to pass to the vents 824. In embodiments, the device 800 can mitigate against fluid breakthrough past the filter 816 due to the inclusion of the pad 828. In embodiments, the inclusion of the pad 828 can reduce the risk of contamination of the fluid from the outside environment via the vents 824 by providing an additional barrier between outside environment and the fluid.

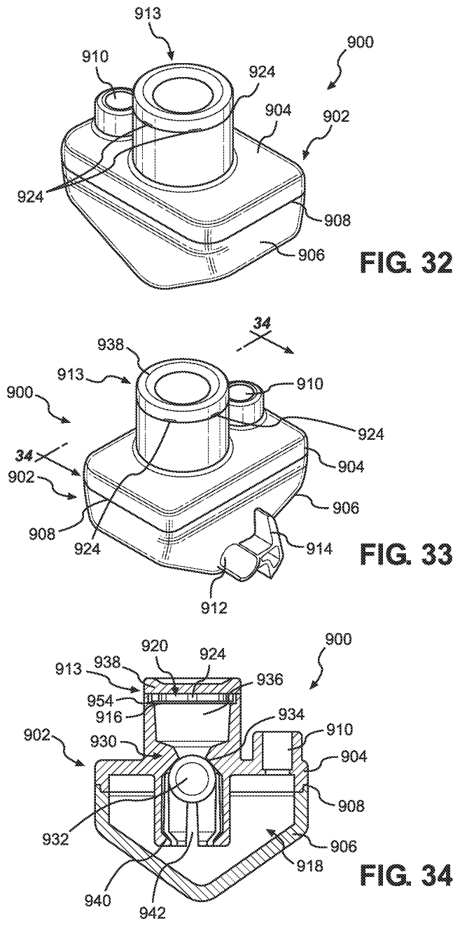

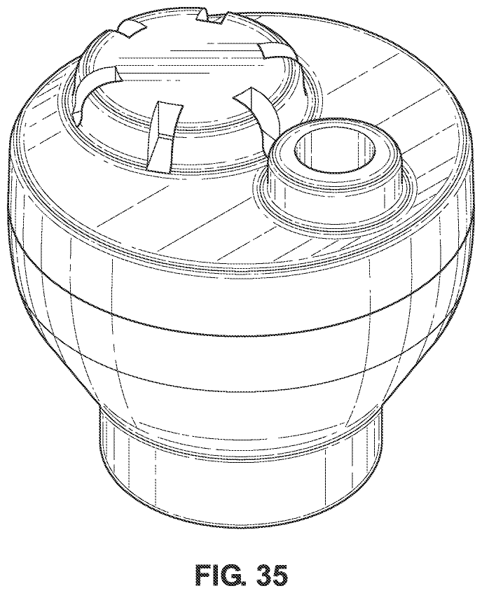

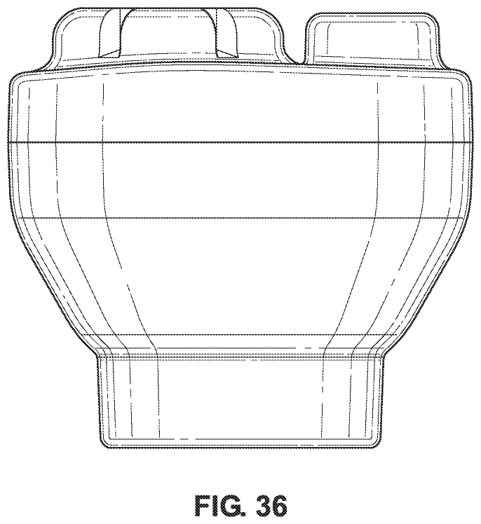

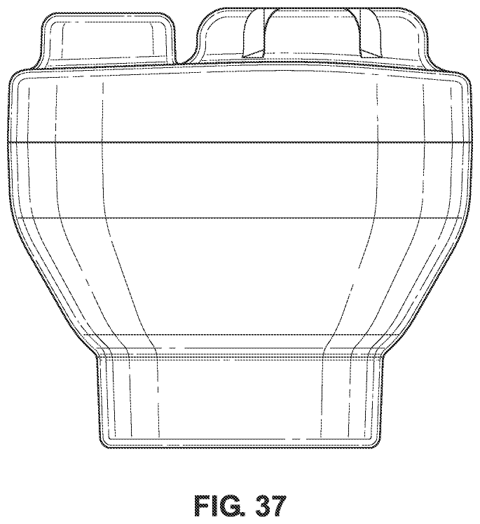

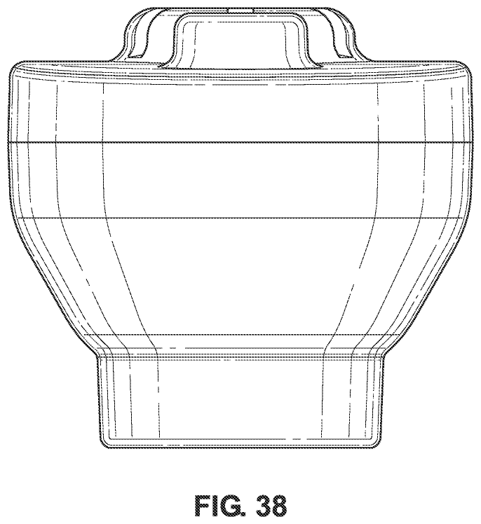

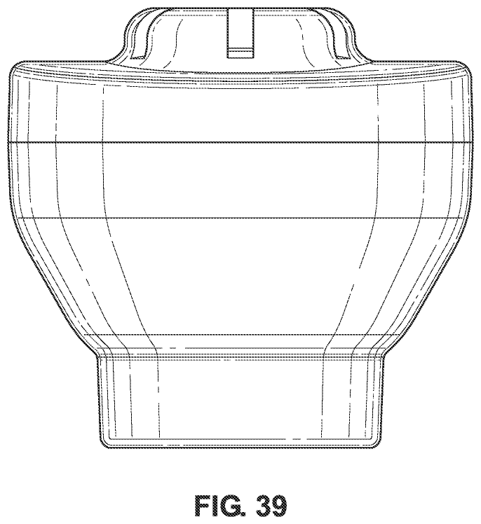

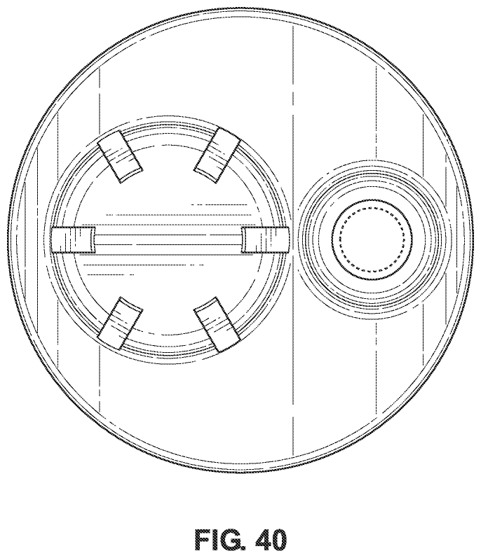

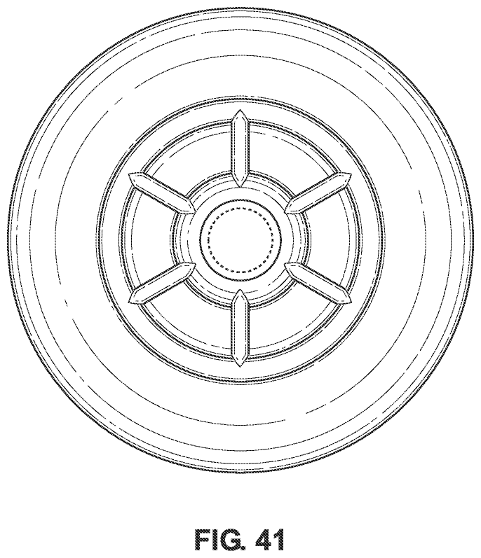

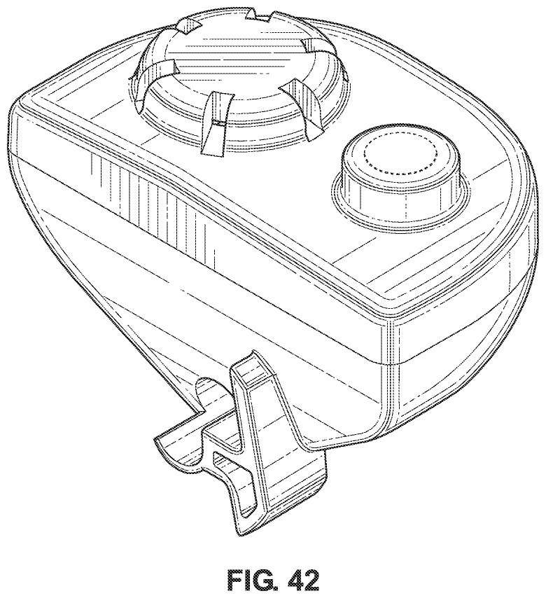

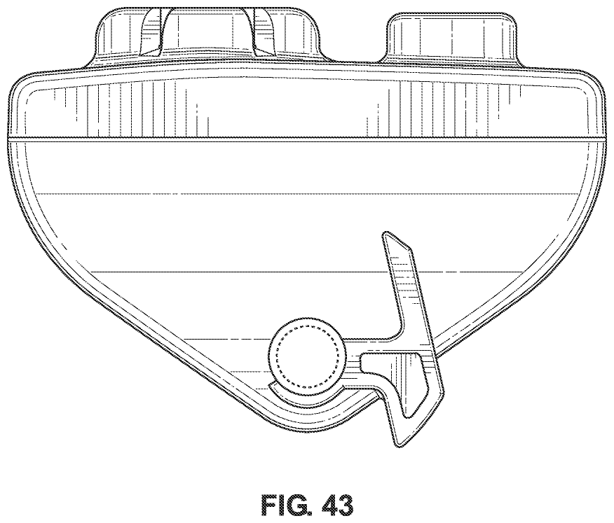

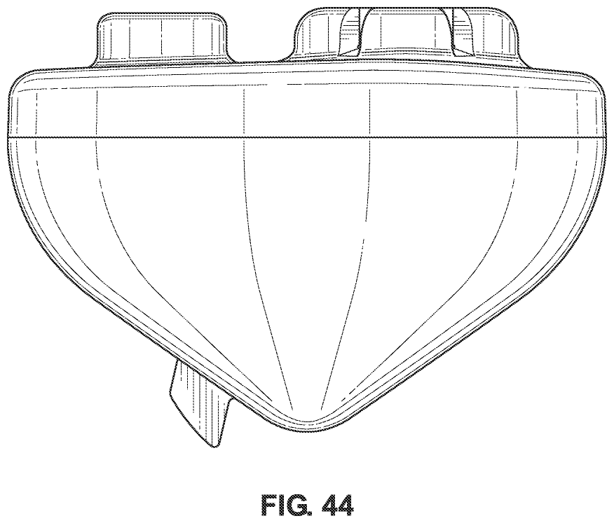

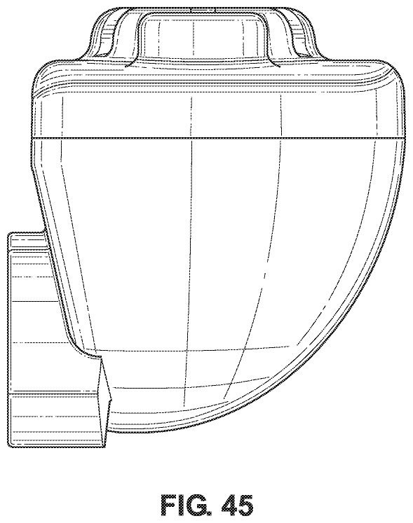

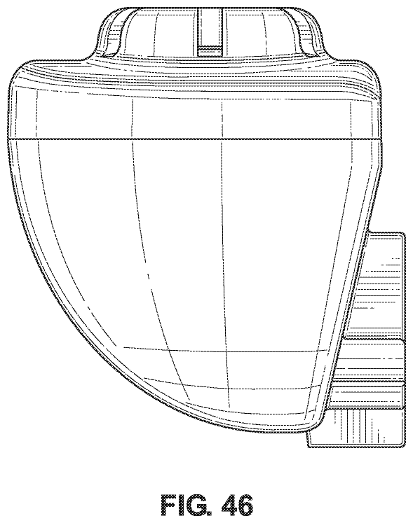

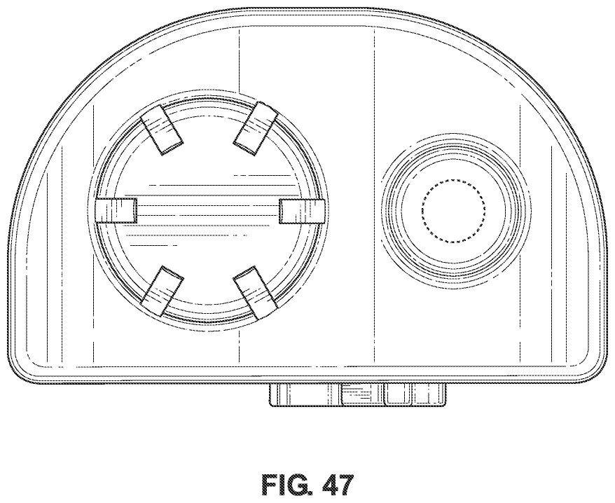

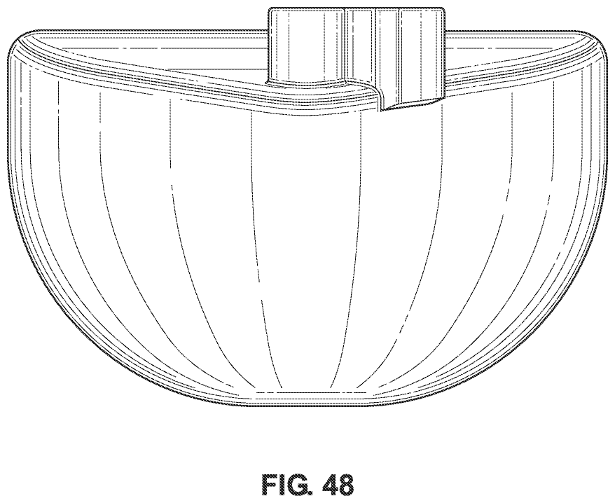

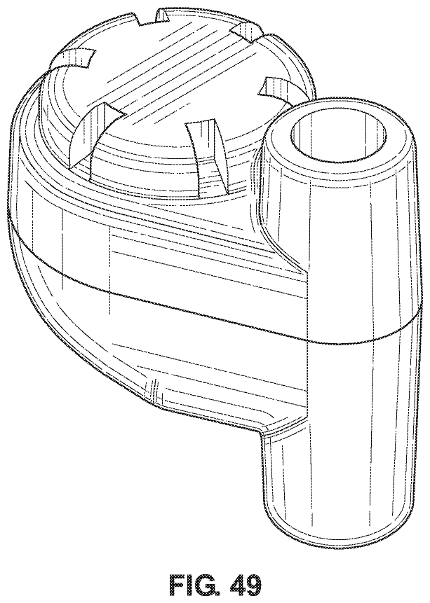

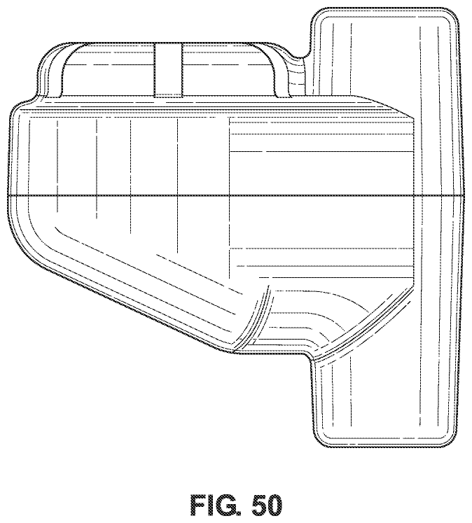

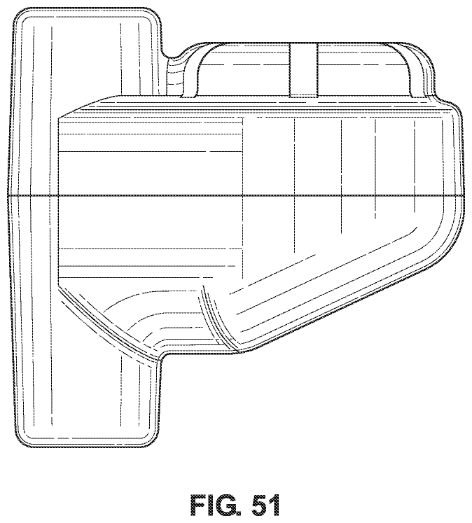

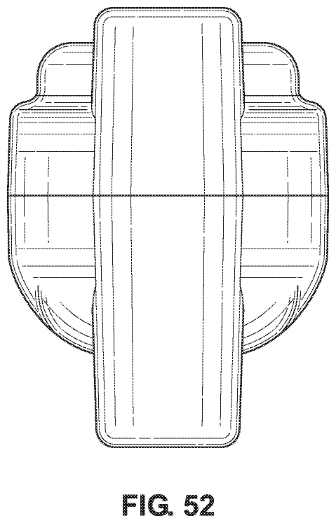

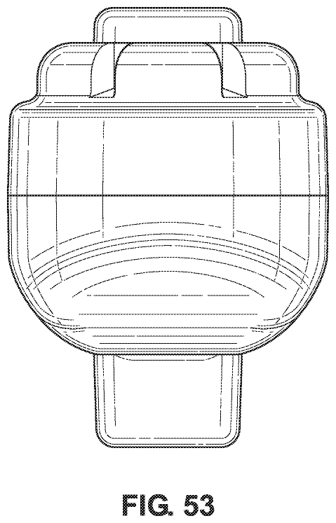

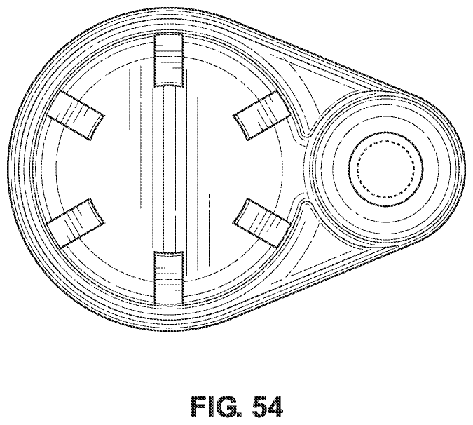

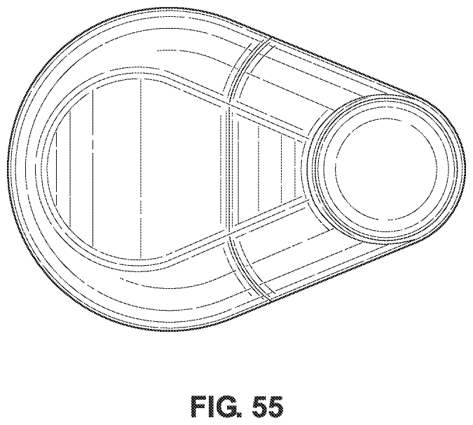

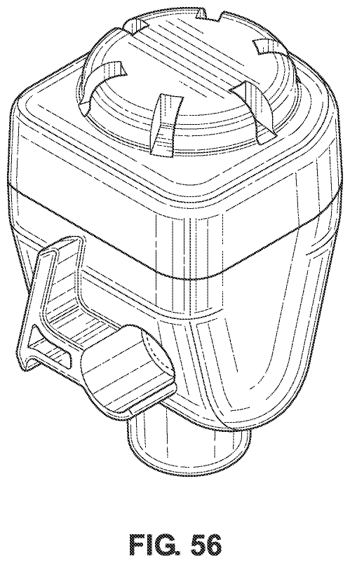

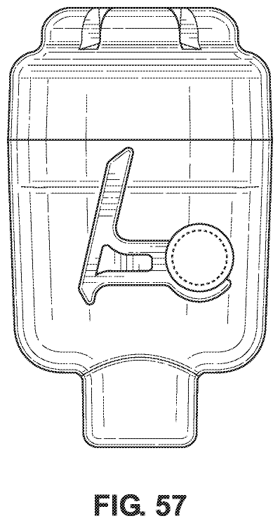

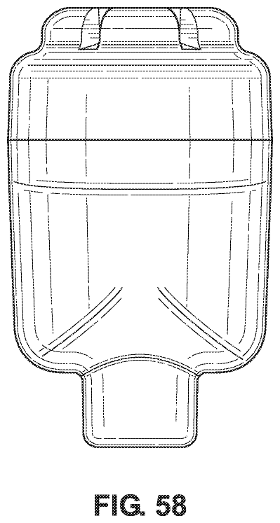

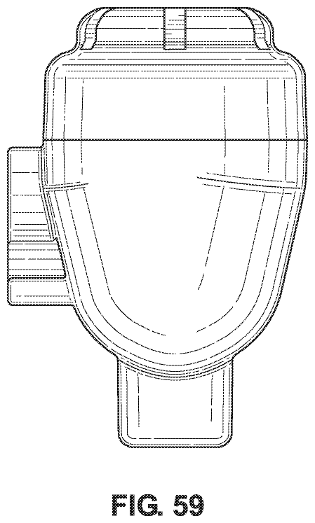

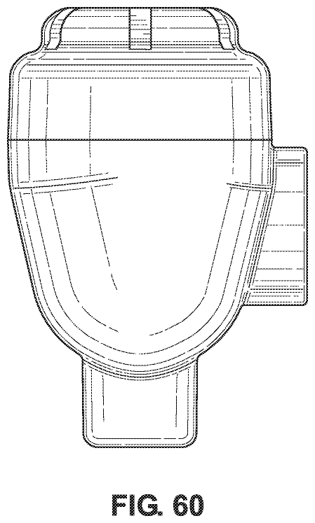

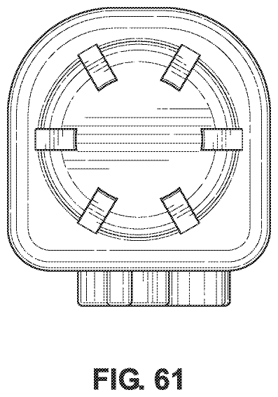

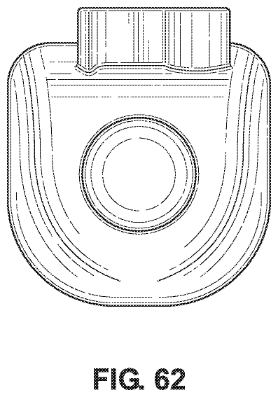

FIGS. 32-34 show views of an eighth embodiment of the device 900 for removing a gas (e.g., air) from a fluid according to aspects of the invention. FIG. 32 shows a perspective view of the device 900. FIG. 33 shows another perspective view from an outlet-side of the device 900. FIG. 34 shows a cross-section view along section line 34-34 of the device 900 shown in FIG. 33. Except such structures, features, relationships, and/or functionalities that persons of skill in the art would recognize as clearly mutually exclusive, the device 900 can include any of the structures, features, relationships, and/or functionalities of the devices 100, 200, 300, 400, 500, 600, 700, and vice versa. For example and as shown in FIGS. 32-34, the device 900 can include any or all of a body 902, a first portion 904, a second portion 906, an interface 908, an inlet 910, a fluid outlet 912, a gas outlet 913, a connector 914, a filter 916, a first chamber 918, a second chamber 920, and vents 924, which can include any of the features, relationships, and/or functionalities described previously with respect to like structures of the devices 200, 300, 400, 500, 600, and/or 700. The device 900 can be used with any or all of the fluid delivery systems 1, 2, 3, or 4 to remove gas from a fluid flowing through any of the respective fluid delivery systems 1, 2, 3, or 4.

The device 900 can include a valve 930 that can selectively isolate the filter 916 from the fluid flowing through the first chamber 918, as previously described. For example, the valve 930 can be configured to automatically isolate the filter 916 from the fluid in the first chamber 918 when the fluid fills a predetermined volume of the first chamber 918 and/or when the fluid exerts a predetermined pressure on the valve 930. The valve 930 can be interposed between the first chamber 918 and the filter 916. At least a portion of the valve 930 can be integrally formed with the body 902. Additionally, or alternatively some or all of the components of the valve 930 can be distinct from the body 902 and can be joined to the body via known techniques.

As shown for example in FIG. 34, in embodiments the valve 930 can include a ball 932 and a seat 934. The ball 932 can be configured to float when immersed in the fluid (e.g., water). For example, the ball 932 can be formed of a lightweight material such as a plastic (e.g., polypropylene), and/or the ball 932 can be hollow.

When fluid within the first chamber 918 is below a predetermined level, the ball 932 can be disengaged from the seat 934 such that gas contained within the first chamber 918 can freely flow through an opening in the seat 934 to a third chamber 936 defined by the interior of the body 902 between the first chamber 918 and the second chamber 920. The filter 916 can be arranged between the third chamber 936 and the second chamber 920. For example, in embodiments the body 902 can include a third portion 938 and the filter 916 can be sandwiched the third portion 938 and the first portion 904 at a second interface 954. Alternatively, the filter 916 can be attached to the body using a number of other techniques (e.g., sonic welding), as previously described. When the ball 932 is disengaged from the seat 934, gas can freely exit from the first chamber 918, through the opening in the seat 934, through the third chamber 936, through the filter 916, into the second chamber 920, and out the vents 924, which in embodiments can be formed in the body 902 (e.g., in the first portion 904 and/or in the third portion 938).

When the first chamber 918 fills with the fluid, the ball 932 can rise with the fluid in the first chamber 918 until the ball 932 is pressed by the fluid into sealing contact with the seat 934. When the ball 932 is pressed into sealing contact with the seat 934 fluid communication between the first chamber 918 and structures beyond the seat 934 (i.e., the third chamber 936, the filter 916, the second chamber 920, the vents 924, etc.) can be interrupted. This can prevent the fluid from exerting pressure on the filter 916 and improve longevity of the filter 916. The device 900 can separate gas from the fluid even when the valve 930 is closed by trapping the gas in a top of the first chamber 918. In embodiments, the valve 930 can include a track 940 that restrains lateral movement of the ball 932 to guide the ball 932 towards the seat 934 as the fluid level rises. The track 940 can include slits 942 to permit fluid flow, e.g., gas flow, from the first chamber 918 and through the seat 934 when the ball 932 is disengaged from the seat 934 so that gas can be evacuated from the first chamber 918.

In embodiments, components of the valve 930 can be integral with components of the body 902. For example, any or all of the seat 934, the third chamber 936, the third portion 938, the track 940, among others, can be integral with the body 902. Any or all components of the valve 930 can be distinct from the body 902 and can be joined with the body 902 via any number of known techniques.

In embodiments, the device 900 can intermittently remove gas from the fluid through the vents 924. For example, the device 900 can remove gas from the fluid through the vents 924 when the valve 930 is open and can prevent removal of gas from the fluid through the vents 924 when the valve is closed. In embodiments, the device 900 can mitigate against fluid breakthrough past the filter 916 due to the inclusion of the valve 930. In embodiments, the valve 930 can reduce or prevent degradation of the filter 916 by limiting contact with, for example, certain caustic medicines such as chemotherapy drugs within the fluid. In embodiments, the device 900 can hold between 1.2 mL and 1.8 mL of gas, though the device 900 can remove more gas by venting the gas through the vents 924.

It will be appreciated that the foregoing description provides examples of the invention. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

1. A device for separating a gas from a fluid, the device comprising:
a filter;
a body defining an interior, the filter being within the interior and segmenting the interior into a first chamber and a second chamber;
an inlet that is configured to communicate the fluid into the first chamber;
a first outlet that is configured to communicate the fluid out of the first chamber; and
a second outlet that is adjacent the filter and configured to communicate the gas out of the second chamber,
wherein the second outlet has a closed central portion and defines a plurality of vents spaced apart along a periphery of the second outlet,
wherein each of the plurality of vents is defined by two side walls and a bottom surface that slopes downwardly away from the closed central portion,
wherein the filter is impermeable to a liquid of the fluid and is configured to isolate the second chamber from the liquid of the fluid when the fluid is in the first chamber, and
wherein the filter is permeable to the gas of the fluid and is configured to permit communication of the gas from the first chamber to the second chamber to separate the gas from the liquid of the fluid.

2. The device of claim 1, wherein the inlet and the second outlet extend from a top of the body.

3. The device of claim 2, wherein a center of each the inlet and the second outlet are each laterally offset from a center of the top of the body.

4. The device of claim 1, wherein the first outlet has an inner diameter and the second outlet has an inner diameter, and
wherein the inner diameter of the first outlet is less than the inner diameter of the second outlet.

5. The device of claim 1, wherein an inner surface of a top of the body tapers towards the second outlet.

6. The device of claim 1, wherein an inner surface of a bottom of the body tapers towards the first outlet.

7. (canceled)

8. The device of claim 1, wherein the body comprises projections extending into the second chamber to resist a pressure exerted by the fluid on the filter.

9. The device of claim 8, wherein the projections extend radially inwardly into the second chamber to the closed central portion.

10. The device of claim 8, wherein the projections segment the second chamber into a plurality of sub-chambers.

11. The device of claim 10, wherein each vent of the plurality of vents is defined within one respective sub-chamber of the plurality of sub-chambers.

12. The device of claim 1, wherein the first outlet of the device is configured to connect the device to an inlet of a pump.

13. The device of claim 1, wherein the inlet of the device is configured to connect the device to an outlet of a pump.

14. The device of claim 1, wherein the filter comprises at least one of PTFE or acrylic copolymer matrix.

15. The device of claim 1, wherein:
the filter is a first filter,
the device further comprises a second filter held within the second chamber,
the second filter is impermeable to the liquid of the fluid, and
the second filter is permeable to the gas of the fluid.

16. The device of claim 1, further comprising a pad within the second chamber, wherein the pad is configured to absorb the liquid and the pad is configured to be permeable to the gas.

17. The device of claim 1, further comprising a valve that is configured to selectively isolate the filter from the fluid in the first chamber when at least one of the fluid fills a volume of the first chamber or the fluid exerts a predetermined pressure on the valve.

18-30. (canceled)

31. The device of claim 1, wherein the plurality of vents are spaced apart around a circumference of the second outlet.

32. The device of claim 1, wherein the plurality of vents comprise six vents.

33. The device of claim 1, wherein the closed central portion is continuously closed.

34. The device of claim 1, wherein the filter has pores having a diameter of 0.2 microns.

35. The device of claim 1, wherein the filter is formed of a hydrophobic material or an oleophobic material.

36. The device of claim 1, wherein the second chamber holds between 1 mL and 1.8 mL of the gas.

37. The device of claim 1, wherein the filter is disk shaped.

38. The device of claim 9, wherein the projections intersect within the closed central portion.

39. The device of claim 9, wherein the projections extend perpendicularly from a surface of the filter to the second outlet to isolate each of the plurality of vents from one another.

40. The device of claim 12, wherein:
an inner diameter of the first outlet is greater than or equal to an inner diameter of an inlet supply line that connects the first outlet to the inlet of the pump, or
the inner diameter of the first outlet is greater than or equal to an outer diameter of an outlet supply line that connects the first outlet to the inlet of the pump.

41. The device of claim 13, wherein:
an inner diameter of the inlet is greater than or equal to an inner diameter of an inlet supply line that connects the inlet to the outlet of the pump, or
the inner diameter of the inlet is greater than or equal to an outer diameter of an outlet supply line that connects the inlet to the outlet of the pump.

42. The device of claim 1, wherein the inlet and the first outlet extend along a first axis, and the second outlet extends along a second axis that is parallel to the first axis.

43. The device of claim 1, wherein the inlet extends along a first axis and the first outlet extends along a second axis that is perpendicular to the first axis.

44. The device of claim 1, wherein an inner surface of a bottom of the body is cup-shaped.
